(19) United States
(12) Patent Application Publication
(10) Pub. No.: US 2026/0218184 A1
(43) Pub. Date: Jul. 30, 2026

(54) INDUCTION OF CELLULAR LETHALITY IN CANCER

(71) Applicants: FUNDAÇÃO GIMM - GULBENKIAN INSTITUTE FOR MOLECULAR MEDICINE, Lisboa (PT); FACULDADE DE MEDICINA DA UNIVERSIDADE DE LISBOA, Lisboa (PT)

(72) Inventors: Sérgio Fernandes De Almeida, Lisboa (PT); Cristiana Marisa De Sousa Morgado, Penaverde (PT); Claus Maria Azzalin, Lisboa (PT)

(21) Appl. No.: 19/146,888

(22) PCT Filed: Jan. 10, 2024
(86) PCT No.: PCT/EP2024/050456

§ 371 Date: Jul. 9, 2025

(30) Foreign Application Priority Data
Jan. 10, 2023 (PT) PT118469

(51) Int. Cl. C12N 15/113

(52) U.S. Cl. CPC C12N 15/113; C12N 2310/14

(57) ABSTRACT

The present invention relates to the finding that depletion of a FANC protein or gene, such as FANCM, FANCD2 or FANCI, causes synthetic lethality in SETD2 deficient cells. Methods and compounds for use in the treatment of SETD2 deficient cancers are provided.

### CROSS-REFERENCE TO RELATED APPLICATIONS

This application is a national stage entry under 35 U.S.C. § 371 of International Application No. PCT/EP2024/050456, filed Jan. 10, 2024, which claims the benefit of Portuguese Application No. PT118469, filed Jan. 10, 2023. The applications are incorporated herein by reference in their entirety

### REFERENCE TO ELECTRONIC SEQUENCE LISTING

The contents of the electronic sequence listing (MEWE-139.xml; Size: 14,446 byte; and Date of Creation: Jul. 9, 2025) is herein incorporated by reference in its entirety.

### FIELD

This invention relates to the induction of cellular lethality in cancer cells.

### BACKGROUND

Many conventional anticancer therapies, such as radiotherapy or chemotherapy, target dividing cells and are highly toxic for non-cancer cells, causing serious side-effects.

Therapeutic strategies that are based on synthetic lethal interactions, i.e. the induction of cell death upon the concomitant, but not individual, disruption of two genes, allow tumour cells to be selectively targeted with high efficiency whilst preserving non-cancer cells.

There is a need for novel and improved cancer therapies based on synthetic lethal interactions.

### SUMMARY

The present inventors have found that depletion of Fanconi anaemia, complementation group M protein (FANCM), for example by siRNA, is selectively lethal to cancer cells that are deficient in SETD2. FANCM antagonists may therefore be useful in the treatment of SETD2 deficient cancers.

A first aspect of the invention provides a method of treatment of a cancer in an individual in need thereof comprising; reducing FANCM expression or activity in the individual, wherein the cancer is SETD2 deficient.

A second aspect of the invention provides a FANCM antagonist for use in a method of treatment of a SETD2 deficient cancer.

A third aspect of the invention provides the use of a FANCM antagonist in the manufacture of a medicament for treatment of a SETD2 deficient cancer.

A fourth aspect provides a method of screening for a compound that induces cell death in SETD2 deficient cancer cells comprising determining the binding of a test compound to FANCM, wherein binding to FANCM is indicative that the compound induces cells death in SETD2 deficient cells

A fifth aspect provides a method of screening for a compound that induces cell death in SETD2 deficient cancer cells comprising determining the effect of a test compound on the expression or activity of FANCM, wherein reduction in expression or activity of FANCM is indicative that the compound induces cell death in SETD2 deficient cancer cells.

A sixth aspect provides a method of determining responsiveness of a cancer in an individual to treatment with a FANCM antagonist comprising determining the presence of one or more SET2D deficient cancer cells in a sample obtained from the individual, the presence of one or more SETD2 deficient cancer cells in the sample being indicative that the cancer is responsive to the treatment with the FANCM antagonist.

A seventh aspect provides method of selecting an individual having a SETD2 deficient cancer for treatment with a FANCM antagonist, the method comprising; identifying a cancer cell obtained from the individual as deficient in SETD2 relative to normal cells.

Other aspects and embodiments of the invention are described in more detail below.

### BRIEF DESCRIPTION OF THE FIGURES

FIG. 1 shows a reduction in cell culture density of SETD2 deficient clear cell carcinoma cells transfected with siRNAs to deplete FANCM. Wild-type (WT) or CRISPR/Cas9 SETD2-Knockout (KO) clear cell renal cell carcinoma cells (Caki2 cell line) were transfected with control (siCtrl) or FANCM siRNAs. (A) Shows cell culture density 3 days after knockdown of FANCM. (B) Shows a reduction in cell culture density in SETD2-KO Caki cells 6 after knockdown.

FIG. 2 shows increased cell death in SETD2 deficient clear cell carcinoma cells 3 days after transfection with siRNAs to deplete FANCM. WT or SETD2-KO clear cell carcinoma cells (786-0 cell line) were transfected with control or FANCM siRNAs. After 3 days in culture (A), a viability assay was carried out by measuring levels of propidium iodide staining in SETD2-KO 786-0 cells by flow cytometry, and (B) shows the percentage of PI+ cells in WT or SETD2-KO clear cell carcinoma cells (786-0 cell line) transfected with siCtrl or siFANM. P values were calculated with a two-tailed Student's t-test. P<0.05, **P<0.005, ***P<0.001.

FIG. 3 shows increased cell death in SETD2 deficient clear cell carcinoma cells 6 days after transfection with siRNAs to deplete FANCM. WT or SETD2-KO clear cell carcinoma cells (786-0 cell line) were transfected with control or FANCM siRNAs. After 6 days in culture (A), a viability assay was carried out by measuring levels of propium iodide staining in SETD2-KO 786-0 cells by flow cytometry, and (B) shows the percentage of PI+ cells in WT or SETD2-KO clear cell carcinoma cells (786-0 cell line) transfected with siCtrl or siFANCM. P values were calculated with a two-tailed Student's t-test. P<0.05, **P<0.005, ***P<0.001.

FIG. 4 shows increased cell death in SETD2 deficient cells transfected with siRNAs to deplete FANGD2. WT (AGHN and Gaki-1) or SETD2-KO cells (RGG-AB, RGG-ER, RGG-MF) were transfected with control or FANGM siRNAs. The percentage of PI+ cells in WT or SETD2-KO cells transfected with siGtrl or siFANGD3 is shown after 3 days (A) and 6 days (B). P values were calculated with a two-tailed Student's t-test. P<0.05, **P<0.005, ***P<0.001.

### DETAILED DESCRIPTION

This invention relates to the finding that reduction or abolition of FANCM expression or activity in SETD2 deficient cancer cells induces cell death. This may be useful in the treatment of cancer conditions.

Su(var), Enhancer of zeste, Trithorax (SET)-domain containing 2 (SETD2; EC:2.1.1.359; Gene ID:29072) is a histone methyltransferase responsible for trimethylation on residues lysine 36 on histone H3 (i.e., H3K36me3). SETD2-mediated H3K36me3 is deposited during transcriptional elongation and is important in maintaining nucleosome occupancy, DNA double-stranded break repair, RNA splicing and transcription termination (Li J, et al. SETD2: an epigenetic modifier with tumour suppressor functionality. Oncotarget. 2016; 7(31):50719-50734.; de Almeida S F, Carmo-Fonseca M. Design principles of interconnections between chromatin and pre-mRNA splicing. Trends Biochem Sci. 2012; 37(6):248-253.; Carvalho S, et al. Histone methyltransferase SETD2 coordinates FACT recruitment with nucleosome dynamics during transcription. Nucleic Acids Res. 2013; 41(5):2881-2893.; Carvalho S, et al. SETD2 is required for DNA double-strand break repair and activation of the p53-mediated checkpoint. Elife. 2014; 3:e02482.; Grosso A R, et al.) Pervasive transcription read-through promotes aberrant expression of oncogenes and RNA chimeras in renal carcinoma. Elife. 2015; 4:e09214. Loss of SETD2 therefore causes genomic instability and SETD2 is mutated across a range of human cancers (Fahey C C, et al. Cold Spring Harb Perspect Med. 2017; 7(5):a026468).

A SETD2 deficient cancer may comprise or consist of one or more cancer cells which have a reduced or abolished expression and/or activity of SETD2. Expression and/or activity of SETD2 may be reduced or abolished in the cancer cells by means of one or more variations in one or both alleles of the SETD2 gene, such as one or more mutations, translocations or deletions, or by means of epigenetic modifications that impair SETD2 expression or activity, for example altered DNA or histone methylation, or by means of mutation(s) in a gene encoding a regulatory factor, for example HOX transcript antisense RNA.

Mutations may include nonsynonymous mutations, including frameshift, missense, nonsense, splice site, nonstop and translation start site changes, truncating mutations, including nonsense, nonstop, frameshift deletion, frameshift insertion, and splice sites; and inframe mutations, including inframe deletions and inframe insertions. Mutations in the gene encoding SETD2 may result in the production of a variant polypeptide with altered activity, such as reduced or abolished activity. Genomic translocations or chromosomal rearrangements (such as inversions) involving the SETD2 gene and/or deletion of part or all of the SET2D gene may also result in loss of functional SETD2 protein. Epigenetic changes, for example DNA methylation or histone (de)acetylation and (de)methylation, may also cause reduced or abolished SETD2 activity.

The presence of one or more variations in one or both alleles of the SETD2 gene may be determined by detecting, in one or more cells of a test sample obtained from an individual, the presence of a nucleic acid sequence from the SETD2 gene which comprises the one or more mutations, or by detecting the variant polypeptide which is encoded by the gene.

Various methods are available for determining the expression and/or activity of SETD2 in a sample obtained from an individual.

Genetic testing, such as DNA sequencing, exome-sequencing, comparative genomic hybridization, in situ hybridization (e.g. fluorescence in situ hybridisation (FISH)), PCR, RT-PCR, karyotype analysis, classical and molecular cytogenetics, or molecular testing such as enzymatic activity assays, complementation assays, SETD2 or H3K36me3 western blot or immunofluorescence, bisulfite sequencing may reveal one or more variations present in the SETD2 gene (including mutation of one or more nucleotides, deletions, translocations, inversions, chromosomal rearrangements, or epigenetic alterations) or deficient SETD2 protein production or activity.

Nucleic acid, which may be genomic DNA, RNA or cDNA, or an amplified region thereof, may be sequenced to identify or determine the presence of one or more mutations therein. A mutation may be identified by comparing the sequence obtained with the database sequence of the component, as set out above. In particular, the presence of one or more mutations that cause abrogation or loss of function of SETD2 may be determined. The presence of one or more of the type of mutations described above is indicative of reduced or abolished activity.

Sequencing may be performed using any one of a range of standard techniques. Sequencing of an amplified product may, for example, involve precipitation with isopropanol, resuspension and sequencing using a TaqFS+ Dye terminator sequencing kit. Extension products may be electrophoresed on an ABI 377 DNA sequencer and data analysed using Sequence Navigator software.

A specific amplification reaction such as polymerase chain reaction (PCR) using one or more pairs of primers may conveniently be employed to amplify the region of interest within the nucleic acid sequence, for example, the portion of the sequence suspected of containing mutations or polymorphisms. The amplified nucleic acid may then be sequenced as above, and/or tested in any other way to determine the presence or absence of a mutation or polymorphism which reduces or abolishes the expression and/or activity of SETD2.

Furthermore, having sequenced nucleic acid of an individual or sample, the sequence information can be retained and subsequently searched without recourse to the original nucleic acid itself. Thus, for example, scanning a database of sequence information using sequence analysis software may identify a sequence alteration or mutation.

In some embodiments, a cancer may be identified as deficient in SETD2 by assessing the level of expression or activity of SET2D. Expression levels may be determined, for example, by Western Blot, ELISA, RT-PCR, nucleic acid hybridisation or karyotypic analysis.

Mutations and polymorphisms associated with a SETD2 deficient cancer may also be detected at the protein level by detecting the presence of a variant polypeptide, for example by immunohistochemistry.

Furthermore, SETD2 is the only enzyme responsible for H3K36me3 (Edmunds et al., (2008) (The EMBO journal 27(2):406-420)) so determining levels H3K36me3 in a sample obtained from an individual may be used to identify a cancer as SETD2 deficient. Levels of H3K36me3 may be measured using immunofluorescence or western blot. Reduced levels of the trimethylation mark H3K36me3 are indicative of reduced or abolished SETD2 activity and/or expression.

A sample may be a sample of cancer cells and may be obtained from an individual using conventional techniques.

Fanconi anemia group M protein (FANCM) is an ATP-dependent DNA helicase/translocase (EC 3.6.4.13) involved in homologous recombination, meiosis and DNA repair. FANCM may be human FANCM. The human gene (Gene ID: 57697) encoding the FANCM polypeptide has 25 exons and is located at 14q21.2. A reference human FANCM amino acid sequence is shown in SEQ ID NO: 1. Other reference human FANCM amino acid sequences have the database accession numbers NP_001295062.1, NP_1295063.1, and NP_065988.1. A FANCM polypeptide as described herein may comprise a reference amino acid sequence, such as SEQ ID NO: 1 or a variant thereof.

A variant thereof as used herein is an amino acid sequence having at least 80% sequence identity, at least 85% sequence identity, at least 90% sequence identity, at least 95% identity, or at least 98% identity to a reference amino acid sequence, such as SEQ ID NO: 1.

A reference human FANCM coding nucleotide sequence is shown in SEQ ID NO: 2. Other reference human FANCM coding sequences have the database accession numbers NM_001308133.1, NM_001308134.1 and NM_020937.4. A FANCM nucleotide sequence as described herein may comprise a nucleotide sequence of a reference human FANCM coding sequence, such as SEQ ID NO: 2 or a variant thereof.

Sequence identity is commonly defined with reference to the algorithm GAP (Wisconsin GCG package, Accelerys Inc, San Diego USA). GAP uses the Needleman and Wunsch algorithm to align two complete sequences that maximizes the number of matches and minimizes the number of gaps. Generally, default parameters are used, with a gap creation penalty=12 and gap extension penalty=4. Use of GAP may be preferred but other algorithms may be used, e.g. BLAST (which uses the method of Altschul et al. (1990) J. Mol. Biol. 215: 405-410), FASTA (which uses the method of Pearson and Lipman (1988) PNAS USA 85: 2444-2448), SSEARCH (Smith and Waterman (1981) J. Mol Biol. 147:195-197;), HMMER3 (Johnson L S et al BMC Bioinformatics. 2010 Aug. 18; 11( ):431) or the TBLASTN program, of Altschul et al. (1990) supra, generally employing default parameters (see for example Pearson Curr Prot Bioinformatics (2013) Chapt 3 Uniy 3.1 doi:10.1002/0471250953.bi0301s42). In particular, the psi-Blast algorithm may be used (Altschul et al. Nucl. Acids Res. (1997) 25 3389-3402). Sequence identity and similarity may also be determined using Genomequest™ software (Gene-IT, Worcester MA USA). Sequence comparisons are preferably made over the full-length of the relevant sequence described herein.

In other embodiments, for example when the individual to be treated is a non-human mammal, FANCM may be non-human mammalian FANCM. Reference non-human FANCM amino acid and coding sequences are available on public databases.

A reduction in FANCM expression or activity is shown herein to reduce the viability of SETD2 deficient cancer cells.

FANCM activity may be reduced in an individual by administering a FANCM antagonist that reduces FANCM expression or activity. A FANCM antagonist may inhibit the activity of FANCM, for example a FANCM inhibitor, or may reduce or suppress the expression of FANCM, for example a suppressor nucleic acid or targeted nuclease.

FANCM inhibitors may, for example, include small chemical molecules, for example non-polymeric organic compounds having a molecular weight of 900 Daltons or less. Suitable small molecule FANCM inhibitors may, for example inhibit ATP binding to the ATPase domain of FANCM; DNA binding to the translocase domain of FANCM and/or FANCM binding to a binding partner, such as MHF, FAAP24, BLM, RMI, Topo Illa. Suitable techniques for the rational design of small molecule inhibitors through structural analysis of FANCM are well-known in the art.

FANCM inhibitors may, for example, include biological molecules that specifically bind to FANCM. In some embodiments, a biological molecule may specifically bind to the DEAH helicase-like domain corresponding to residues 83-591 of SEQ ID NO: 1.

Biological molecules may include peptides. Peptides may comprise or consist of from 5 to 40 amino acids, for example, from 6 to 10 amino acids and may be derived from FANCM or binding partners thereof as described above. Biological molecules may also include antibodies, antibody fragments and antibody derivatives and non-immunoglobulin binding molecules, such as aptamers, trinectins, anticalins, kunitz domains, transferrins, nurse shark antigen receptors and sea lamprey leucine-rich repeat proteins. Suitable techniques for the generation of biological molecules that specifically bind to FANCM are well known in the art.

The terms “FANCM antagonist” and “FANCM inhibitor” as used herein, cover pharmaceutically acceptable salts and solvates of these compounds.

Suitable antagonists for reducing or suppressing FANCM expression include suppressor nucleic acids, targetable nucleases and nucleic acids encoding such agents. Nucleic acid encoding a suppressor nucleic acid or targetable nuclease may be contained in a vector. Suitable expression vectors are well-known in the art and include viral vectors, such as retroviral, adenoviral, adeno-associated viral, lentiviral, vaccinia or herpes vectors.

The expression of active FANCM protein may be reduced by a suppressor nucleic acid or targetable nuclease compared with a control cell or may be absent i.e. the transcription of the FANCM gene and/or translation of FANCM mRNA may be reduced or absent, such that the cell treated with the suppressor nucleic acids or targetable nuclease lacks or has a reduced amount of active FANCM protein compared to a control cell. Reducing the amount of active FANCM protein to 20% of the amount in control cells or lower is shown to be sufficient to induce cell death. For example, a cell may express up to 5%, up to 10%, up to 15% or up to 20%, of the active FANCM polypeptide that is expressed by control cells.

In some embodiments, nucleic acid suppression may be used to reduce the expression of active FANCM polypeptide. The use of nucleic acid suppression techniques such as anti-sense and RNAi suppression, to down-regulate expression of target genes is well-established in the art.

Cells may be transfected with a suppressor nucleic acid (i.e. a nucleic acid molecule which suppresses FANCM expression), such as an siRNA or shRNA, or a heterologous nucleic acid encoding the suppressor nucleic acid. The suppressor nucleic acid reduces the expression of active FANCM polypeptide by interfering with transcription and/or translation, thereby reducing FANCM activity in the cells.

RNAi involves the expression or introduction into a cell of an RNA molecule which comprises a sequence which is identical or highly similar to the FANCM coding sequence. The RNA molecule interacts with mRNA which is transcribed from the FANCM gene, resulting in the sequence specific degradation or specific post-transcriptional gene silencing (PTGS) of the mRNA. This reduces or suppresses expression of active FANCM polypeptide (Angell & Baulcombe (1997) The EMBO Journal 16, 12:3675-3684; Voinnet & Baulcombe (1997) Nature 389: pg 553).

The RNA molecule is preferably double stranded RNA (dsRNA) (Fire A. et al Nature 391, (1998)). Synthetic siRNA duplexes have been shown to specifically suppress expression of endogenous and heterologous genes in a wide range of mammalian cell lines (Elbashir S M. et al. Nature, 411, 494-498, (2001)).

Suitable RNA molecules for use in RNAi suppression include short interfering RNA (siRNA). siRNA are double stranded RNA molecules of 15 to 40 nucleotides in length, preferably 15 to 28 nucleotides or 19 to 25 nucleotides in length, for example 19, 20, 21, 22, 23, 24 or 25 nucleotides in length. For example, two unmodified 21 mer oligonucleotides may be annealed together to form a siRNA. A siRNA molecule may contain a 3′ and/or 5′ overhang on each strand having a length of about 0, 1, 2, 3, 4, or 5 nucleotides. The overhang lengths of the strands are independent, i.e., the length of the overhang on one strand is not dependent on the length of the overhang on the second strand.

Other suitable RNA molecules for use in RNAi include small hairpin RNAs (shRNAs). shRNA are single-chain RNA molecules which comprise or consist of a short (e.g. 19 to 25 nucleotides) antisense nucleotide sequence, followed by a nucleotide loop of 5 to 9 nucleotides, and the complementary sense nucleotide sequence (e.g. 19 to 25 nucleotides). Alternatively, the sense sequence may precede the nucleotide loop structure and the antisense sequence may follow. The nucleotide loop forms a hairpin turn which allows the base pairing of the complementary sense and antisense sequences to form the shRNA.

A suppressor nucleic acid, such as a siRNA or shRNA, may comprise or consist of a sequence which is identical or substantially identical (i.e. at least 90%, at least 95% or at least 98% identical) to all or part (for example, 15 to 40 nucleotides) of a reference FANCM nucleotide coding sequence, such as SEQ ID NO: 2, or its complement. Suitable reference sequences coding FANCM that may be used for the design of suppressor nucleic acids are publicly available and include SEQ ID NO: 2. FANCM activity is suppressed in the cancer cells by down-regulation of the production of active FANCM polypeptide by the suppressor nucleic acid. For example, a siRNA to suppress the expression of human FANCM may comprise 18 to 22 contiguous nucleotides from SEQ ID NO: 2.

Examples of preferred siRNA molecules for the suppression of human FANCM include SEQ ID NO: 3 (siFa) and SEQ ID NO: 4 (siFb). Suitable siRNA molecules have also been described in Silva B, et al., Nat Commun. 2019; 10(1):2253. siRNA molecules for the suppression of human FANCM are also commercially available (siRNA IDs s33621 and s33619, ThermoFisher) and have been described in Lu R, et al. Nat Commun. 2019; 10(1):2252. [published correction appears in Nat Commun. 2019 Nov. 20; 10(1):5345].

Suppressor nucleic acids, such as siRNAs and shRNAs, for reducing FANCM expression may be readily designed using reference FANCM coding sequences and software tools which are widely available in the art and may be produced using routine techniques. For example, a suppressor nucleic acid may be chemically synthesized; produced recombinantly in vitro or cells (Elbashir, S. M. et al., Nature 411:494-498 (2001); Elbashir, S. M., et al., Genes & Development 15:188-200 (2001)) or obtained from commercial sources (e.g. Cruachem (Glasgow, UK), Dharmacon Research (Lafayette, Colo., USA)).

In some embodiments, two or more suppressor nucleic acids may be used to suppress the expression of FANCM. For example, a pool of siRNAs may be employed. Suitable siRNAs and siRNA pools may be produced using standard techniques.

Nucleic acid suppression may also be carried out using anti-sense techniques. Anti-sense oligonucleotides may be designed to hybridise to the complementary sequence of nucleic acid, pre-mRNA or mature mRNA, interfering with the production of the base excision repair pathway component so that its expression is reduced or completely or substantially completely prevented. In addition to targeting coding sequence, anti-sense techniques may be used to target control sequences of a gene, e.g. in the 5′ flanking sequence, whereby the anti-sense oligonucleotides can interfere with expression control sequences. The construction of anti-sense sequences and their use is well known in the art (Peyman and Ulman, Chemical Reviews, 90:543-584, (1990); Crooke, Ann. Rev. Pharmacol. Toxicol. 32:329-376, (1992)).

Anti-sense oligonucleotides may be generated in vitro or ex vivo for administration or anti-sense RNA may be generated in vivo within the cancer cells in which down-regulation of FANCM is desired. Thus, double-stranded DNA may be placed under the control of a promoter in a “reverse orientation” such that transcription of the anti-sense strand of the DNA yields RNA which is complementary to normal mRNA transcribed from the sense strand of the target gene. The complementary anti-sense RNA sequence is thought then to bind with mRNA to form a duplex, inhibiting translation of the endogenous mRNA from the target gene into protein.

The complete sequence corresponding to the FANCM coding sequence in reverse orientation need not be used. For example, fragments of sufficient length may be used. It is a routine matter for the person skilled in the art to screen fragments of various sizes and from various parts of the coding or flanking sequences of a gene to optimise the level of anti-sense inhibition. It may be advantageous to include the initiating methionine ATG codon, and perhaps one or more nucleotides upstream of the initiating codon. A suitable fragment may have about 14-23 nucleotides, e.g. about 15, 16 or 17.

In other embodiments, targeted mutagenesis may be used to reduce the expression of active FANCM polypeptide. The use of targeted mutagenesis techniques such as gene editing, to knock out or abolish expression of target genes is well-established in the art (see for example Gaj et al (2013) Trends Biotechnol. 31(7) 397-405).

One or more mutations, such as insertions, substitutions, or deletions, may be introduced into the FANCM gene in the cancer cells. Suitable mutations include deletions of all or part of the FANCM gene, for example, one, two or more exons, frameshift mutations, or nonsense mutations introducing premature stop codons. In some preferred embodiments, one or more premature stop codons may be introduced into the FANCM coding sequence. Preferably, mutations, such as premature stop codons, are introduced into the first 400 codons of the coding sequence, to eliminate the ATPase domain of FANCM. The mutations may prevent the expression of active FANCM polypeptide, for example by impairing transcription or translation of the FANCM gene or causing an inactive polypeptide to be expressed.

Targeted mutagenesis to introduce one or more mutations may be performed by any convenient method. For example, the cancer cells may be transfected with a heterologous nucleic acid which encodes a targetable nuclease. The targetable nuclease may inactivate the FANCM gene encoding FANCM in one or more cells of the individual, for example, by introducing one or more mutations that prevent the expression of active FANCM polypeptide.

The targetable nuclease may inactivate the FANCM gene encoding FANCM selectively in cancer cells of the individual. The targetable nuclease may be targeted specifically to cancer cells by conventional techniques, including cell targeted delivery vehicles, such as viral vectors that express a ligand for a specific cell type; direct administration of the targetable nuclease to a tumour e.g. by injection; or the expression of the targetable nuclease from heterologous nucleic acid selectively in cancer cells, for example using a tissue specific promoter.

The targetable nuclease may be site-specific (e.g. ZFN or TALEN) or may be expressed with one or more targeting sequences that target the nuclease to the FANCM gene (e.g. CRISPR/Cas).

The heterologous nucleic acid encoding the targetable nuclease may include an inducible promoter that promotes expression of the targetable nuclease and optional targeting sequence within a specific cell type, for example a tumour cell. For example, the inducible promoter could be a promoter-enhancer cassette that selectively favours expression of the targetable nuclease and the optional targeting sequence within the tumour cell over other types of host cells.

Suitable targeting nucleases include, for example, site-specific nucleases, such as zinc-finger nucleases (ZFNs), transcription activator-like effector nucleases (TALENs), and meganucleases or RNA guided nucleases, such as clustered regularly interspaced short palindromic repeat (CRISPR) nucleases.

Zinc-finger nucleases (ZFNs) comprise one or more Cys₂-His₂ zinc-finger DNA binding domains and a cleavage domain (i.e., nuclease). The DNA binding domain may be engineered to recognize and bind to any nucleic acid sequence using conventional techniques (see for example Qu et al. (2013) Nucl Ac Res 41(16):7771-7782). The use of ZFNs to introduce mutations into target genes is well-known in the art (see for example, Beerli et al Nat. Biotechnol. 2002; 20:135-141; Maeder et al Mol. Cell. 2008; 31:294-301; Gupta et al Nat. Methods. 2012; 9:588-590) and engineered ZFNs are commercially available (Sigma-Aldrich (St. Louis, MO).

Transcription activator-like effector nucleases (TALENs) comprise a nonspecific DNA-cleaving nuclease fused to a DNA-binding domain comprising a series of modular TALE repeats linked together to recognise a contiguous nucleotide sequence. The use of TALEN targeting nucleases is well known in the art (e.g. Joung & Sander (2013) Nat Rev Mol Cell Bio 14:49-55; Kim et al Nat Biotechnol. (2013); 31:251-258. Miller J C, et al. Nat. Biotechnol. (2011) 29:143-148. Reyon D, et al. Nat. Biotechnol. (2012); 30:460-465).

Meganucleases are endodeoxyribonucleases characterized by a large recognition site (double-stranded DNA sequences of 12 to 40 base pairs); as a result this site generally occurs only once in any given genome (see for example Silva et al. (2011) Curr Gene Ther 11(1):11-27).

CRISPR targeting nucleases (e.g. Cas9) complex with a guide RNA (gRNA) to cleave genomic DNA in a sequence-specific manner. The crRNA and tracrRNA of the guide RNA may be used separately or may be combined into a single RNA to enable site-specific mammalian genome cutting within the FANCM gene or its regulatory elements. The use of CRISPR/Cas9 systems to introduce insertions or deletions into genes as a way of decreasing transcription is well known in the art (see for example Cader et al Nat Immunol 2016 17 (9) 1046-1056, Hwang et al. (2013) Nat. Biotechnol 31:227-229; Xiao et al., (2013) Nucl Acids Res 1-11; Horvath et al., Science (2010) 327:167-170; Jinek M et al. Science (2012) 337:816-821; Cong L et al. Science (2013) 339:819-823; Jinek M et al. (2013) eLife 2:e00471; Mali P et al. (2013) Science 339:823-826; Qi L S et al. (2013) Cell 152:1173-1183; Gilbert L A et al. (2013) Cell 154:442-451; Yang H et al. (2013) Cell 154:1370-1379; and Wang H et al. (2013) Cell 153:910-918).

In some embodiments, the targetable nuclease is a Cas endonuclease, preferably Cas9, which is expressed in the cancer cells in combination with a guide RNA targeting sequence that targets the Cas endonuclease to cleave genomic DNA within the FANCM gene and generate insertions or deletions that prevent expression of active FANCM polypeptide.

Nucleic acid sequences encoding a suppressor nucleic acid or targetable nuclease and optionally a guide RNA may be comprised within an expression vector. Suitable vectors can be chosen or constructed, containing appropriate regulatory sequences, including promoter sequences, terminator fragments, polyadenylation sequences, enhancer sequences, marker genes and other sequences as appropriate. Preferably, the vector contains appropriate regulatory sequences to drive the expression of the encoding nucleic acid in a host cell. Suitable regulatory sequences to drive the expression of heterologous nucleic acid coding sequences in a range of expression systems are well-known in the art and include constitutive promoters, for example viral promoters such as CMV or SV40. In some preferred embodiments, a tissue-specific or inducible promoter, such as a photoinducible promoter, may be employed to selectively express the suppressor nucleic acid or targetable nuclease and optionally guide RNA in cancer cells. A vector may also comprise sequences, such as origins of replication and selectable markers, which allow for its selection and replication and expression in bacterial hosts, such as E. coli and/or in eukaryotic cells, such as yeast, insect or mammalian cells. Vectors suitable for use in expressing a suppressor nucleic acid or targetable nuclease in mammalian cells include plasmids and viral vectors e.g. retroviruses, lentiviruses, adenoviruses, and adeno-associated viruses. Suitable techniques for expressing a suppressor nucleic acid or targetable nuclease in mammalian cells are well known in the art (see for example; Molecular Cloning: a Laboratory Manual: 3rd edition, Russell et al., 2001, Cold Spring Harbor Laboratory Press or Protocols in Molecular Biology, Second Edition, Ausubel et al. eds. John Wiley & Sons, 1992; Recombinant Gene Expression Protocols Ed RS Tuan (March 1997) Humana Press Inc).

Other aspects of the invention relate to the use of FANCM to screen for compounds that increase cell death or reduce viability of SETD2 deficient cells and are potentially useful in the treatment of SETD2 deficient cancers.

Screening methods may be used to identify test compounds that bind to an isolated FANCM protein. A method of screening for a compound that reduces viability or increases cell death in SETD2 deficient cells may comprise determining the binding of a test compound to FANCM. For example, a test compound may be contacted with FANCM and the binding of the test compound to FANCM determined. Binding between the test compound and FANCM may be indicative that the test compound reduces viability or increases cell death in SETD2 deficient cells.

The binding of a test compound to FANCM may be determined by standard techniques, such as surface plasmon resonance (SPR).

In some embodiments, the ability of a test compound to inhibit the interaction of FANCM with a binding partner may be determined. A method of screening for a compound that reduces viability or increases cell death in SETD2 deficient cells may comprise determining the effect of a test compound on the binding of FANCM to a binding partner. For example, FANCM may be contacted with a binding partner in the presence and absence of a test compound. A reduction in binding between FANCM and the binding partner in the presence relative to the absence of test compound may be indicative that the test compound reduces viability or increases cell death in SETD2 cells.

Binding partners are proteins that naturally bind to FANCM within a cell, for example during homologous recombination, meiosis and DNA repair. FANCM binding partners may include MHF, FAAP24, HCLK2, the BTR complex and individual members of the BTR complex, such as BLM, RMI1, RMI2 and Topo Illa.

Screening methods may be used to identify test compounds that inhibit the activity of FANCM. A method of screening for a compound that reduces viability or increases cell death in SETD2 cells may comprise determining the effect of a test compound on the activity of FANCM. For example, the activity of FANCM may be determined in the presence and absence of a test compound. A decrease in activity of FANCM in the presence relative to the absence of the test compound being indicative that the compound reduces viability or increases cell death in SETD2 deficient cells.

In some embodiments, the ATP-dependent DNA helicase/translocase activity of the FANCM may be determined in the presence relative to the absence of test compound. A decrease or reduction in ATP-dependent DNA helicase/translocase activity in the presence of the test compound may be indicative that the test compound inhibits the activity of FANCM protein. For example, the test compound may be a FANCM inhibitor. Suitable methods of determining activity, including ATPase and translocase assays, are well known in the art. For example, ATP hydrolysis may be measured in the presence of fork-structured DNA as described in Coulthard R, et al., Structure. 2013; 21(9):1648-1658. A reduction of ATP hydrolysis may be indicative of the test compound decreasing or reducing FANCM DNA helicase/translocase activity. Translocase activity may be determined using a D-loop dissociation assay, which tests D-loop dissociation catalysed by FANCM, and is described in Gari K, Décaillet C, Delannoy M, Wu L, Constantinou A. Proc Natl Acad Sci USA. 2008; 105(42):16107-16112. Decreased or abolished D-loop dissociation in the presence of a test compound indicates that the test compound may reduce or inhibit the translocase activity of FANCM.

The precise format of any of the screening or assay methods of the present invention may be varied by those of skill in the art using routine skill and knowledge. The skilled person is well aware of the need to employ appropriate control experiments.

FANCM for use in screening methods may be an isolated polypeptide comprising the full-length FANCM sequence, for example a FANCM reference sequence, such as SEQ ID NO:1 as set out herein, or a variant or fragment thereof. Suitable fragments may include at least 50, at least 100 or at least 150 contiguous amino acids from a FANCM reference sequence. In some embodiments, FANCM fragments comprising the ATPase or translocase activity may be employed, for example a fragment comprising or consisting of the N terminal DEAH helicase-like domain corresponding to residues 83-591 of SEQ ID NO: 1. Isolated FANCM polypeptides may be produced using standard recombinant techniques.

A test compound may be an isolated molecule or may be comprised in a sample, mixture or extract, for example, a biological sample. Compounds which may be screened using the methods described herein may be natural or synthetic chemical compounds used in drug screening programmes and may include, for example, small organic molecules, polypeptides and nucleic acids, such as aptamers. Extracts of plants, microbes or other organisms, which contain several characterised or uncharacterised components may also be used.

Suitable test compounds for screening include compounds that inhibit similar activities to the ATP-dependent DNA helicase/translocase activity of FANCM. For example, suitable test compounds may be ATP analogues.

Suitable test compounds may be produced using rational drug design to provide test candidate compounds with particular molecular shape, size and charge characteristics suitable for modulating FANCM activity.

Combinatorial library technology provides an efficient way of testing a potentially vast number of different compounds for ability to modulate FANCM activity. Such libraries and their use are known in the art, for all manner of natural products, small molecules and peptides, among others. The use of peptide libraries may be preferred in certain circumstances. In some embodiments, libraries of biological molecules, such as aptamers or antibody molecules.

The amount of test compound which may be added to an assay of the invention will normally be determined by trial and error depending upon the type of compound used. Typically, from about 0.001 nM to 1 mM or more concentrations of putative inhibitor compound may be used, for example from 0.01 nM to 100 pM, e.g. 0.1 to 50 pM, such as about 10 pM. Even a compound which has a weak effect may be a useful lead compound for further investigation and development.

Test compounds may include peptides derived from FANCM or binding partners thereof as described above. Membrane permeable peptide fragments of from 5 to 40 amino acids, for example, from 6 to 10 amino acids may be tested for their ability to bind to FANCM or inhibit its activity. The modulatory properties of a peptide above may be increased by the addition of one of the following groups to the C terminal: chloromethyl ketone, aldehyde and boronic acid. These groups are transition state analogues for serine, cysteine and threonine proteases. The N terminus of a peptide fragment may be blocked with carbobenzyl to inhibit aminopeptidases and improve stability (Proteolytic Enzymes 2nd Ed, Edited by R. Beynon and J. Bond, Oxford University Press, 2001).

Test compounds may include antibodies, antibody fragments and antibody derivatives and non-immunoglobulin binding molecules, such as aptamers, trinectins, anticalins, kunitz domains, transferrins, nurse shark antigen receptors and sea lamprey leucine-rich repeat proteins. Suitable molecules may be directed to the DEAH helicase-like domain corresponding to residues 83-591 of SEQ ID NO: 1 or another part of the FANCM protein. Candidate modulatory antibody molecules may be characterised and their binding regions determined to provide single chain antibodies and fragments thereof which are responsible for inhibiting activity or blocking interactions with binding partners. Suitable antibodies may be obtained using techniques which are standard in the art, including, for example immunising a mammal with a suitable peptide, such as a fragment of the pro-inflammatory polypeptide, or isolating a specific antibody from a recombinantly produced library of expressed immunoglobulin variable domains, e.g. using lambda bacteriophage or filamentous bacteriophage which display functional immunoglobulin binding domains on their surfaces; for instance see WO92/01047.

Aptamers directed to FANCM are also putative agents for modulating FANCM. Aptamers are nucleic acids that specifically bind to a target molecule. Typically aptamers are small nucleic acids ranging from 15-50 bases in length that fold into defined secondary and tertiary structures, such as stem-loops or G-quartets. Aptamers can bind very tightly with k_{d} for the target molecule of less than 10-¹² M. Aptamers may bind FANCM with a very high degree of specificity. For example, aptamers have been isolated that have greater than a 10000 fold difference in binding affinities between a target molecule and another molecule that differ at only a single position on the molecule. An aptamer may have a k_{d} with FANCM of at least 10, 100, 1000, 10,000, or 100,000 fold lower than the k_{d} with a control polypeptide. The production and use of aptamers is well known in the art (see for example Bunka et al Curr Opin Pharmacol 2010 10 (5) 557-562).

A test compound identified as inhibiting FANCM activity may be investigated further using one or more secondary screens. A secondary screen may involve testing for a biological function or activity in vitro and/or in vivo, e.g. in an animal model. For example, the ability of a test compound to reduce viability or increase cell death of SETD2 cells may be determined. In some embodiments, a secondary screen may involve determining the selectivity of a compound for FANCM by screening against a panel of isolated enzymes.

The effect of a test compound identified as an FANCM inhibitor may be determined in vitro on mammalian cells. For example, the effect of the test compound on an SETD2 deficient cell line may be determined. Increased cell death in the presence relative to the absence of the compound may be indicative that the compound displays an activity useful in the treatment of SETD2 deficient cancer.

Following identification of a FANCM inhibitor that is potentially useful in the treatment of SETD2 deficient cancer as described herein, a method may further comprise modifying the compound to optimise its pharmaceutical properties. Suitable methods of optimisation, for example by structural modelling, are well known in the art. Further optimisation or modification can then be carried out to arrive at one or more final compounds for in vivo or clinical testing.

A test compound identified as a FANCM inhibitor may be isolated and/or purified or alternatively, it may be synthesised using conventional techniques of recombinant expression or chemical synthesis. Furthermore, it may be manufactured and/or used in preparation, i.e. manufacture or formulation, of a composition such as a medicament, pharmaceutical composition or drug. Methods described herein may thus comprise formulating the test compound in a pharmaceutical composition with a pharmaceutically acceptable excipient, vehicle or carrier for therapeutic application.

Whilst a FANCM antagonist as described above, such as a FANCM inhibitor, suppressor nucleic acid, targetable nuclease, nucleic acid encoding a suppressor nucleic acid or targetable nuclease, may be administered alone, the FANCM antagonist will usually be administered in the form of a pharmaceutical composition, which may comprise at least one component in addition to the active agent. A FANCM antagonist may be admixed with other reagents, such as buffers, carriers, diluents, preservatives and/or pharmaceutically acceptable excipients in order to produce a pharmaceutical composition for use in cancer immunotherapy. Suitable reagents are described in more detail below.

Aspects of the invention provide (i) a pharmaceutical composition comprising a FANCM antagonist selected from (a) a FANCM inhibitor (b) FANCM suppressor nucleic acid, (c) FANCM targetable nuclease, (d) nucleic acid encoding a FANCM suppressor nucleic acid or targetable nuclease, and a pharmaceutically acceptable excipient and (ii) a method of producing a pharmaceutical composition for use in cancer immunotherapy, for example to treat a SETD2 deficient cancer, comprising admixing a FANCM antagonist as described above with a pharmaceutically acceptable excipient.

The term “pharmaceutically acceptable” as used herein pertains to compounds, materials, compositions, and/or dosage forms which are, within the scope of sound medical judgement, suitable for use in contact with the tissues of a subject (e.g., human) without excessive toxicity, irritation, allergic response, or other problem or complication, commensurate with a reasonable benefit/risk ratio. Each carrier, excipient, etc. must also be “acceptable” in the sense of being compatible with the other ingredients of the formulation.

Pharmaceutical compositions suitable for administration (e.g. by infusion), include aqueous and non-aqueous isotonic, pyrogen-free, sterile injection solutions which may contain anti-oxidants, buffers, preservatives, stabilisers, bacteriostats, and solutes which render the formulation isotonic with the blood of the intended recipient; and aqueous and non-aqueous sterile suspensions which may include suspending agents and thickening agents. Examples of suitable isotonic vehicles for use in such formulations include Sodium Chloride Injection, Ringer's Solution, or Lactated Ringer's Injection. Suitable vehicles can be found in standard pharmaceutical texts, for example, Remington's Pharmaceutical Sciences, 18^{th} edition, Mack Publishing Company, Easton, Pa., 1990.

A FANCM antagonist or the pharmaceutical composition comprising the FANCM antagonist as described herein may be administered to a subject by any convenient route of administration, whether systemically/peripherally or at the site of desired action, including but not limited to; parenteral, for example, by infusion, including intravenous infusion, in particular intravenous bolus infusion. Suitable infusion techniques are known in the art and commonly used in therapy (see, e.g., Rosenberg et al., New Eng. J. of Med., 319:1676, 1988).

Reducing the activity or expression of FANCM in SETD2 expressing cells has no effect on cell death. The systemic administration of a FANCM antagonist as described herein, for example by oral administration or injection, may therefore elicit a selective cell death effect on SETD2 deficient cancer cells in the individual relative to other non-SETD2 deficient cells in the individual.

It will be appreciated that appropriate dosages of the FANCM antagonist, and compositions comprising the FANCM antagonist, can vary from patient to patient. Determining the optimal dosage will generally involve the balancing of the level of therapeutic benefit against any risk or deleterious side effects of the treatments of the present invention. The selected dosage level will depend on a variety of factors including, but not limited to, the activity of the particular cells, the route of administration, the time of administration, the rate of loss or inactivation of the cells, the duration of the treatment, other drugs, compounds, and/or materials used in combination, and the age, sex, weight, condition, general health, and prior medical history of the patient. The amount of cells and the route of administration will ultimately be at the discretion of the physician, although generally the dosage will be to achieve local concentrations at the site of action which achieve the desired effect without causing substantial harmful or deleterious side-effects.

A typical oral dosage of a small molecule inhibitor is in the range of from about 0.05 to about 1000 mg, preferably from about 0.1 to about 500 mg, and more preferred from about 1.0 mg to about 200 mg administered in one or more dosages such as 1 to 3 dosages. The exact dosage will depend upon the frequency and mode of administration, the sex, age, weight and general condition of the subject treated, the nature and severity of the condition treated and any concomitant diseases to be treated and other factors evident to those skilled in the art. For parenteral routes such as intravenous, intrathecal, intramuscular and similar administration, typically doses are in the order of about half the dose employed for oral administration.

A FANCM antagonist, such as (a) a FANCM inhibitor (b) FANCM suppressor nucleic acid, (c) a FANCM targetable nuclease, or (d) nucleic acid encoding a FANCM suppressor nucleic acid or targetable nuclease, as described herein, may be useful in therapy. For example, a FANCM antagonist which reduces FANCM activity may be administered to an individual for the treatment of a SETD2 deficient cancer.

Methods according to some aspects of the present invention may comprise determining the binding of an oligonucleotide probe to nucleic acid obtained from the sample, for example, genomic DNA, RNA or cDNA. The probe may comprise a nucleotide sequence which binds specifically to a nucleic acid sequence which contains one or more mutations or polymorphisms and does not bind specifically to the nucleic acid sequence which does not contain the one or more mutations or polymorphisms, or vice versa.

The oligonucleotide probe may comprise a label and binding of the probe may be determined by detecting the presence of the label.

A method may include hybridisation of one or more (e.g. two) oligonucleotide probes or primers to a target nucleic acid. Where the nucleic acid is double-stranded DNA, hybridisation will generally be preceded by denaturation to produce single-stranded DNA. The hybridisation may be as part of a PCR procedure, or as part of a probing procedure not involving PCR. An example procedure would be a combination of PCR and low stringency hybridisation.

Binding of a probe to target nucleic acid (e.g. DNA) may be measured using any of a variety of techniques at the disposal of those skilled in the art. For instance, probes may be radioactively, fluorescently or enzymatically labelled. Other methods not employing labelling of probe include examination of restriction fragment length polymorphisms, amplification using PCR, Rnase cleavage and allele specific oligonucleotide probing. Probing may employ the standard Southern blotting technique. For instance, DNA may be extracted from cells and digested with different restriction enzymes. Restriction fragments may then be separated by electrophoresis on an agarose gel before denaturation and transfer to a nitrocellulose filter. Labelled probes may be hybridised to the DNA fragments on the filter and binding determined.

Those skilled in the art are well able to employ suitable conditions of the desired stringency for selective hybridisation, taking into account factors such as oligonucleotide length and base composition, temperature and so on.

Suitable selective hybridisation conditions for oligonucleotides of 17 to 30 bases include hybridization overnight at 42° C. in 6×SSC and washing in 6×SSC at a series of increasing temperatures from 42° C. to 65° C.

Other suitable conditions and protocols are described in Molecular Cloning: a Laboratory Manual: 3^{rd} edition, Sambrook & Russell (2001) Cold Spring Harbor Laboratory Press NY and Current Protocols in Molecular Biology, Ausubel et al. eds. John Wiley & Sons (1992).

Cancer is characterised by abnormal proliferation of malignant cancer cells relative to normal cells and may include leukaemia, such as AML, CML, ALL and CLL, lymphoma, such as Hodgkin lymphoma, non-Hodgkin lymphoma and multiple myeloma, and solid cancers such as sarcomas; skin cancer; melanoma, bladder cancer; brain cancer, such as glioblastoma multiforme, such as paediatric glioblastoma multiforme; breast cancer; uterus cancer; oral cancer; ovary cancer; prostate cancer; lung cancer, such as lung adenocarcinoma, and lung squamous cell carcinoma; colorectal cancer; cervical cancer; liver cancer; head and neck cancer; oesophageal cancer; pancreas cancer; renal cancer, such as clear cell renal cancer; stomach cancer; testicular cancer; cancer of the gall bladder and biliary tracts; thyroid cancer; thymus cancer; cancer of bone; and cerebral cancer. Cancers may be familial or sporadic. A SET2D deficient cancer suitable for treatment as described herein may be a cancer of any of these types that is deficient in SET2D expression or activity.

Other aspects of the invention relate to the identification of individuals with cancer who are suitable for treatment with a FANCM antagonist, such as (a) a FANCM inhibitor, (b) FANCM suppressor nucleic acid, (c) FANCM targetable nuclease, or (d) nucleic acid encoding a FANCM suppressor nucleic acid or targetable nuclease, as described herein. For example, an individual with cancer may be assessed using a method described herein to determine whether treatment with a FANCM antagonist would be likely to be beneficial to the individual i.e. whether the individual is suitable for treatment by a method of the first aspect of the invention. A method of predicting, determining or assessing the responsiveness of a cancer in an individual to a FANCM antagonist which reduces FANCM expression or activity may comprise determining the presence of one or more SET2D deficient cancer cells in a sample of cancer cells from the individual, the presence of one or more SET2D cancer cells in the sample being indicative that the cancer is responsive to said FANCM antagonist.

An individual with a cancer identified as being responsive to a FANCM antagonist may be treated as described herein, for example using a method of the first aspect of the invention.

An individual suitable for treatment with a FANCM antagonist such as (a) a FANCM inhibitor, (b) FANCM suppressor nucleic acid, (c) FANCM targetable nuclease, or (d) nucleic acid encoding a FANCM suppressor nucleic acid or targetable nuclease, as described herein, may be a mammal, such as a rodent (e.g. a guinea pig, a hamster, a rat, a mouse), murine (e.g. a mouse), canine (e.g. a dog), feline (e.g. a cat), equine (e.g. a horse), a primate, simian (e.g. a monkey or ape), a monkey (e.g. marmoset, baboon), an ape (e.g. gorilla, chimpanzee, orang-utan, gibbon), or a human.

In some preferred embodiments, the individual is a human. In other preferred embodiments, non-human mammals, especially mammals that are conventionally used as models for demonstrating therapeutic efficacy in humans (e.g. murine, primate, porcine, canine, or rabbit animals) may be employed.

In some embodiments, the individual may have minimal residual disease (MRD) after an initial cancer treatment.

The term “treatment”, as used herein in the context of treating a condition, pertains generally to treatment and therapy in which some desired therapeutic effect is achieved, for example, the inhibition of the progress of the condition, and includes a reduction in the rate of progress, a halt in the rate of progress and amelioration of the condition, and cure of the condition.

Treatment may be any treatment and therapy, whether of a human or an animal (e.g. in veterinary applications), in which some desired therapeutic effect is achieved, for example, the inhibition or delay of the progress of the condition, and includes a reduction in the rate of progress, a halt in the rate of progress, amelioration of the condition, cure or remission (whether partial or total) of the condition, preventing, delaying, abating or arresting one or more symptoms and/or signs of the condition or prolonging survival of a subject or patient beyond that expected in the absence of treatment.

Treatment as a prophylactic measure (i.e. prophylaxis) is also included. For example, an individual susceptible to or at risk of the occurrence or re-occurrence of cancer may be treated as described herein. Such treatment may prevent or delay the occurrence or re-occurrence of cancer in the individual.

In particular, treatment may include inhibiting cancer growth, including complete cancer remission, and/or inhibiting cancer metastasis. Cancer growth generally refers to any one of a number of indices that indicate change within the cancer to a more developed form. Thus, indices for measuring an inhibition of cancer growth include a decrease in cancer cell survival, a decrease in tumour volume or morphology (for example, as determined using computed tomographic (CT), sonography, or other imaging method), a delayed tumour growth, a destruction of tumour vasculature, improved performance in delayed hypersensitivity skin test, an increase in the activity of cytolytic cancer cells, and a decrease in levels of tumour-specific antigens.

Reduction of FANCM expression or activity is shown herein to have a strong and specific effect on SETD2 deficient cancer cells. In some preferred embodiments, a FANCM antagonist, such as (a) a FANCM inhibitor (b) FANCM suppressor nucleic acid, (c) FANCM targetable nuclease, or (d) nucleic acid encoding a FANCM suppressor nucleic acid or targetable nuclease as described herein may therefore be administered to the individual without other concomitant cancer therapy, such as cytotoxic chemotherapy or radiotherapy i.e. the FANCM antagonist may be administered alone.

In other embodiments, a FANCM antagonist, such as (a) a FANCM inhibitor (b) FANCM suppressor nucleic acid, (c) FANCM targetable nuclease, or (d) nucleic acid encoding a FANCM suppressor nucleic acid or targetable nuclease as described herein may be administered in combination with one or more other therapies, such as cytotoxic chemotherapy or radiotherapy. This may be useful for example in treating cancers that comprise both SETD2 deficient cancer cells and SETD2 positive cancer cells or cancers where the SETD2 status is not determined.

When the FANCM antagonists are used in combination with additional therapeutic agents, the compounds may be administered either sequentially or simultaneously by any convenient route. When a FANCM antagonist is used in combination with an additional therapeutic agent active against the same disease, the dose of each agent in the combination may differ from that when the FANCM antagonists are used alone. Appropriate doses will be readily appreciated by those skilled in the art.

Administration of FANCM antagonists, such as (a) FANCM inhibitors (b) FANCM suppressor nucleic acids (c) FANCM targetable nucleases, or (d) nucleic acids encoding FANCM suppressor nucleic acids or targetable nucleases, as described herein, can be effected in one dose, continuously or intermittently (e.g., in divided doses at appropriate intervals) throughout the course of treatment. Methods of determining the most effective means and dosage of administration are well known to those of skill in the art and will vary with the formulation used for therapy, the purpose of the therapy, the target cell being treated, and the subject being treated. Single or multiple administrations can be carried out with the dose level and pattern being selected by the treating physician.

Other aspects and embodiments of the invention provide the aspects and embodiments described above with the term “comprising” replaced by the term “consisting of” and the aspects and embodiments described above with the term “comprising” replaced by the term “consisting essentially of”.

It is to be understood that the application discloses all combinations of any of the above aspects and embodiments described above with each other, unless the context demands otherwise. Similarly, the application discloses all combinations of the preferred and/or optional features either singly or together with any of the other aspects, unless the context demands otherwise.

Modifications of the above embodiments, further embodiments and modifications thereof will be apparent to the skilled person on reading this disclosure, and as such, these are within the scope of the present invention.

All documents and sequence database entries mentioned in this specification are incorporated herein by reference in their entirety for all purposes.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

Other aspects and embodiments of the invention provide the aspects and embodiments described above with the term “comprising” replaced by the term “consisting of” and the aspects and embodiments described above with the term “comprising” replaced by the term “consisting essentially of”.

The term “downstream” as used herein refers to the 5′ to 3′ direction in a nucleic acid described herein and the term “upstream” as used herein refers to the 3′ to 5′ direction in a nucleic acid described herein

Reference to a nucleotide sequence as set out herein encompasses a DNA molecule with the specified sequence, and encompasses a RNA molecule with the specified sequence in which U is substituted for T, unless context requires otherwise.

It is to be understood that the application discloses all combinations of any of the above aspects and embodiments described above with each other, unless the context demands otherwise. Similarly, the application discloses all combinations of the preferred and/or optional features either singly or together with any of the other aspects, unless the context demands otherwise.

Modifications of the above embodiments, further embodiments and modifications thereof will be apparent to the skilled person on reading this disclosure, and as such, these are within the scope of the present invention.

All documents and sequence database entries mentioned in this specification are incorporated herein by reference in their entirety for all purposes.

“and/or” where used herein is to be taken as specific disclosure of each of the two specified features or components with or without the other. For example, “A and/or B” is to be taken as specific disclosure of each of (i) A, (ii) B and (iii) A and B, just as if each is set out individually herein.

### Experimental

### Simultaneous Loss of FANCM and SET2D Results in Cancer Cell Death

SET Domain-containing 2 (SETD2) is a histone methyltransferase frequently mutated in several types of cancer. The present inventors set out to test the effect of genetic ablation of FANCM on survival of SETD2-deficient cancer cells.

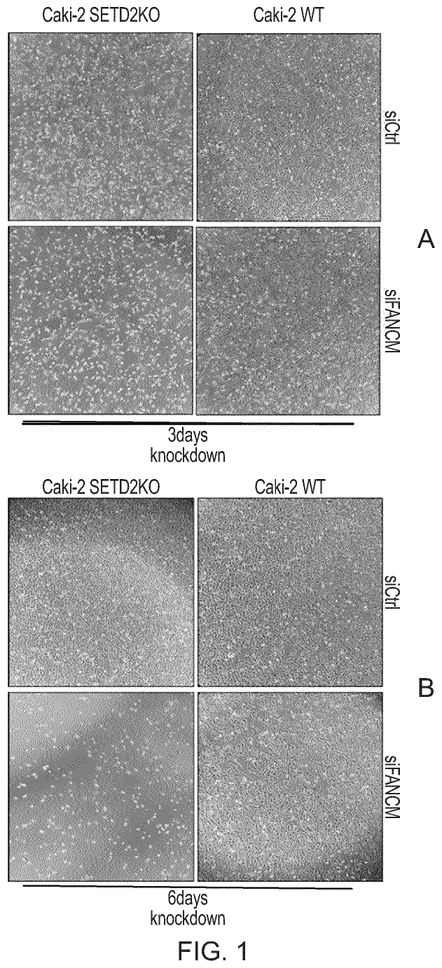

Wild-type (WT) and CRISPR/Cas9 SETD2-Knockout (KO) clear cell renal cell carcinoma cells (Caki2 cell line) were transfected with control (Ctrl) or FANCM siRNAs. After 6 days in culture, SETD2-KO Caki2 cells that were transfected with siRNAs to deplete FANCM show a clear reduction of the cell culture density, suggesting increased cell death in these cells (FIG. 1).

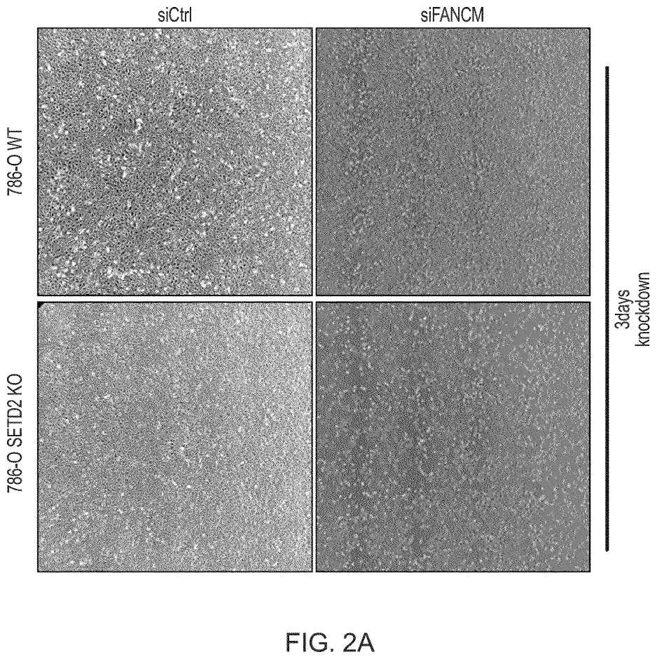

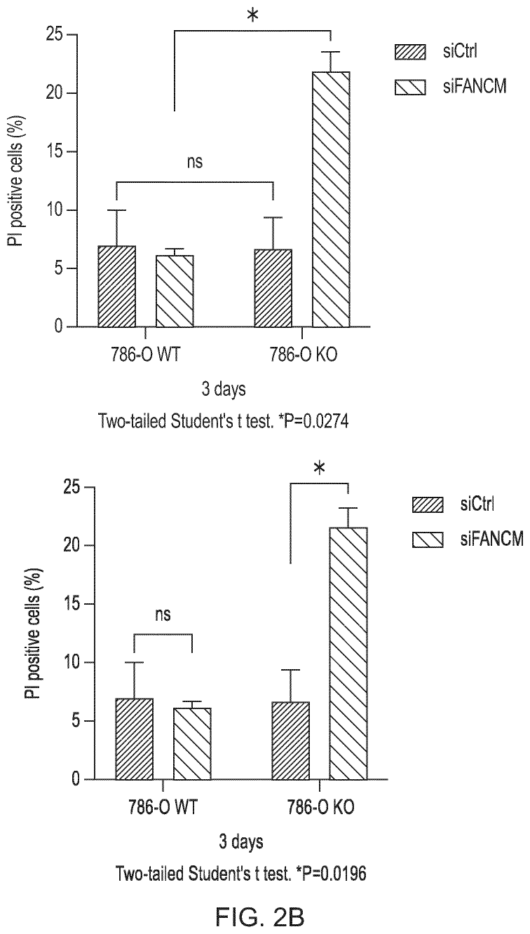

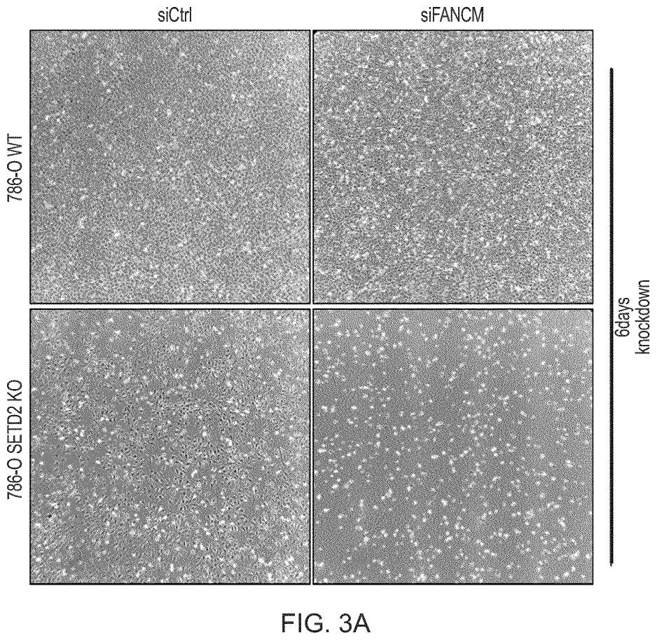

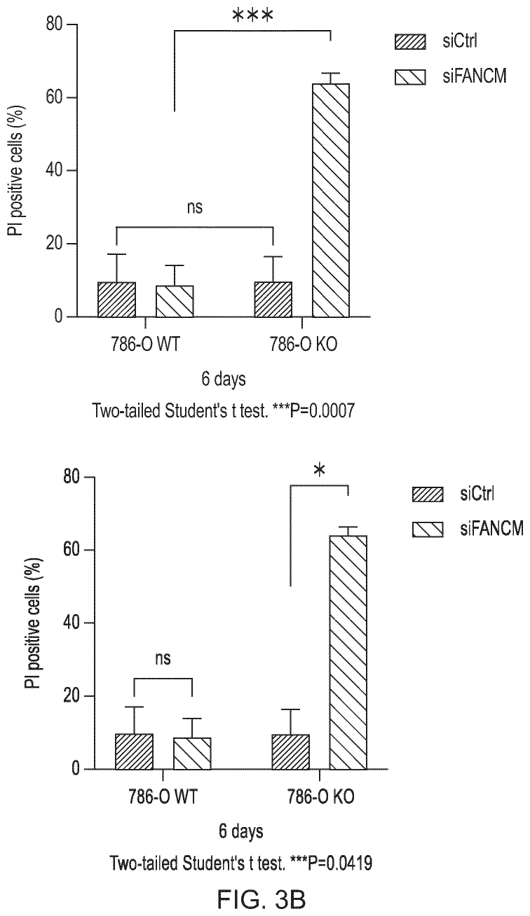

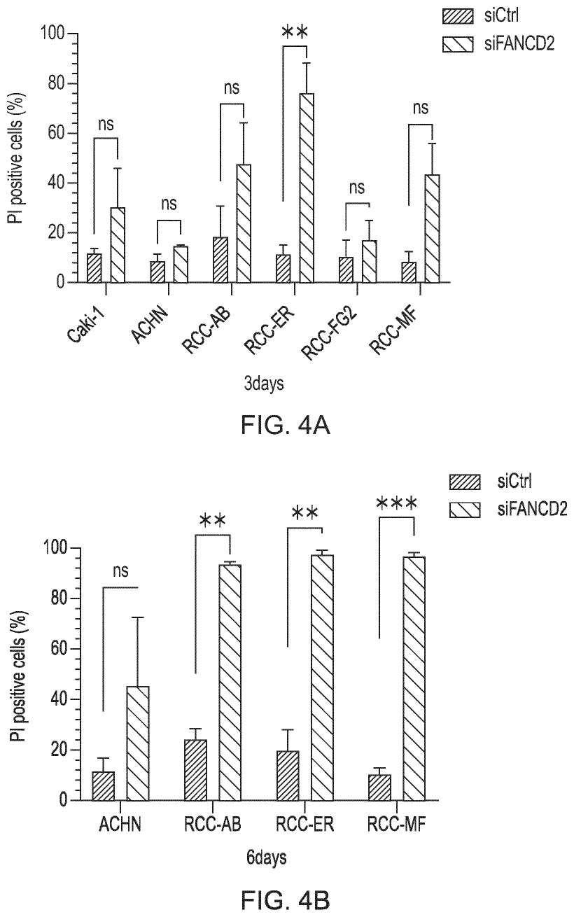

To test whether simultaneous deletion of FANCM and SET2D results in increased cell death, WT and CRISPR/Cas9 SETD2-(KO) clear cell renal cell carcinoma cells (786-0 cell line) were transfected with control (Ctrl) or FANCM siRNAs. As early as 3 days after knockdown, SETD2-KO 786-0 cells that were transfected with siRNAs to specifically deplete FANCM show a significant loss of viability, as revealed by the increased levels of PI staining detected in flow cytometry experiments (FIG. 2). A further increase in cell death specifically in SETD2-KO 786-0 cells that were transfected with siRNAs to specifically deplete FANCM, was observed at day 6 after knockdown (FIG. 3).

These results indicate that the simultaneous loss of FANCM and SETD2 yields a synthetic lethal phenotype that drives cancer cell death. This synthetic lethal phenotype may be exploited as a monotherapy against cancers that have somatic loss of SETD2 or administered in addition to chemotherapy and radiotherapy for the treatment of aggressive or relapsed cancers. It also poses a promising approach for the treatment of metastatic cancer, for which there are few treatment strategies.

Sequences

SEQ ID NO: 1

| | | | | | | |
|---|---|---|---|---|---|---|
| 1 | msgrqrtlfq | twgssisrss | gtpgcssgte | rpqspgsska | plpaaaeaql | esdddvllva |
| 61 | ayeaerqlcl | enggfctsag | alwiyptncp | vrdyqlhisr | aalfcntlvc | lptglgktfi |
| 121 | aavvmynfyr | wfpsgkvvfm | aptkplvtqq | ieacyqvmgi | pqshmaemtg | stqastrkei |
| 181 | wcskrvlflt | pqvmvndlsr | gacpaaeikc | lvideahkal | gnyaycqavq | qvitnlligq |
| 241 | ielrsedspd | iltysherkv | eklivplgee | laaiqktyiq | ilesfarsli | qrnvlmrrdi |
| 301 | pnltkyqiil | ardqfrknps | pnivgiqqgi | iegefaicis | lyhgyellqq | mgmrslyffl |
| 361 | cgimdgtkgm | trsknelgrn | edfmklynhl | ecmfartrst | sangisaiqq | gdknkkfvys |
| 421 | hpklkkleev | viehfkswna | enttekkrde | trvmifssfr | dsvqeiaeml | sqhqpiirvm |
| 481 | tfvghasgks | tkgftqkeql | evvkqfrdgg | yntlvstcvg | eegldigevd | liicfdsqks |
| 541 | pirlvqrmgr | tgrkrqgriv | iilsegreer | iynqsqsnkr | siykaissnr | qvlhfyqrsp |
| 601 | rmvpdginpk | lhkmfithgv | yepekpsrnl | qrkssifsyr | dgmrqsslkk | dwflseeefk |
| 661 | lwnrlyrlrd | sdeikeitlp | qvqfsslqne | enkpagestt | gihqlslsew | rlwqdhplpt |
| 721 | hqvdhsdrcr | hfiglmqmie | gmrheegecs | yelevesylq | medvtstfia | prnesnnlas |
| 781 | dtfithkkss | fikninggss | ssviesdeec | aeivkqthik | ptkivslkkk | vskeikkdql |
| 841 | kkennhgiid | svdndrnstv | enifqedlpn | dkrtsdtdei | aatctinenv | ikepcvllte |
| 901 | cqftnkstss | lagnvldsgy | nsfndeksvs | snlflpfeee | lyivrtddqf | ynchsltkev |
| 961 | lanverflsy | sppplsglsd | leyeiakgta | lenllflpca | ehlrsdkctc | llshsavnsq |
| 1021 | qnlelnslkc | inypsekscl | ydipndnisd | epslcdcdvh | khnqnenlvp | nnrvqihrsp |
| 1081 | aqnlvgennh | dvdnsdlpvl | stdqdeslll | fedvntefdd | vslsplnsks | eslpvsdkta |
| 1141 | isetplvsqf | lisdellldn | nselqdqitr | dansfksrdq | rgvqeekvkn | hedifdcsrd |
| 1201 | lfsvtfdlgf | cspdsddeil | ehtsdsnrpl | ddlygrylei | keisdanyvs | nqaliprdhs |
| 1261 | knftsgtvii | psnedmqnpn | yvhlplsaak | neellspgys | qfslpvqkkv | mstplsksnt |
| 1321 | Insfskirke | ilktpdsske | kvnlqrfkea | Instfdysef | slekskssgp | mylhkschsv |
| 1381 | edgqlltsne | seddeifrrk | vkrakgnvln | spedqknsev | dsplhavkkr | rfpinrsels |
| 1441 | ssdesenfpk | pcsqledfkv | cngnarrgik | vpkrqshlkh | varkflddea | elseedaeyv |
| 1501 | ssdendesen | eqdsslldfl | ndetqlsqai | ndsemraiym | kslrspmmnn | kykmihkthk |
| 1561 | ninifsqipe | qdetyledsf | cvdeeesckg | qsseeevcvd | fnlitddcfa | nskkyktrra |
| 1621 | vmlkemmeqn | cahskkklsr | iilpddssee | ennvndkres | niavnpstvk | knkqqdhcln |
| 1681 | svpsgssaqs | kvrstprvnp | lakqskqtsl | nlkdtisevs | dfkpqnhnev | qsttppfttv |
| 1741 | dsqkdcrkfp | vpqkdgsale | dsstsgascs | ksrphlagth | tslrlpqegk | gtcilvgghe |
| 1801 | itsgleviss | lraihglqve | vcplngcdyi | vsnrmvverr | sqsemlnsvn | knkfieqiqh |
| 1861 | lqsmfericv | ivekdrektg | dtsrmfrrtk | sydsllttli | gagirilfss | cqeetadllk |
| 1921 | elslveqrkn | vgihvptvvn | snksealqfy | lsipnisyit | alnmchqfss | vkrmansslq |
| 1981 | eismyaqvth | qkaeeiyryi | hyvfdiqmlp | ndlnqdrlks | di | |

SEQ ID NO: 2

| | | | | | | |
|---|---|---|---|---|---|---|
| 1 | tgtgcgaagg | aaaccgatgg | ggatcggaac | cgtagcggtt | gagctgctgc | tgctacggat |
| 61 | atctgacaga | agccttcggt | ggttgtcggc | ctaatgagcg | gacggcaaag | aacgcttttt |
| 121 | cagacgtggg | gctcaagtat | ctcccgatca | tctgggactc | cgggttgcag | ctccggaact |
| 181 | gagcgacctc | agagccctgg | cagctccaag | gcgcctttgc | cagcagcage | ggaggctcag |
| 241 | ctggagtcgg | acgatgatgt | gttgcttgtc | gcggcgtacg | aggctgagcg | gcagttgtgt |
| 301 | ctagagaatg | gcgggttctg | cacctccgcg | ggcgccctgt | ggatttaccc | taccaattgc |
| 361 | ccagtgcggg | actaccagct | gcacatttcc | cgggctgctc | tgttttgcaa | tacgctggtg |
| 421 | tgtctgccta | ccggactggg | aaagaccttt | attgccgccg | tggtcatgta | caatttctac |
| 481 | cgctggttcc | cttcaggaaa | ggtggtcttc | atggccccaa | cgaaaccctt | ggtgacacag |
| 541 | cagatcgagg | cttgctacca | ggtgatgggt | atcccgcaat | cccacatggc | cgaaatgaca |
| 601 | gggtctacac | aagcttccac | caggaaggaa | atatggtgca | gtaagagagt | gctttttctt |
| 661 | acacctcagg | tcatggtaaa | tgacctttct | agaggagctt | gtcccgctgc | tgaaataaag |
| 721 | tgtttagtta | ttgatgaagc | tcataaagct | ctcggaaact | atgcttattg | ccaggctgtg |
| 781 | caacaagtta | ttactaacct | gctaattggg | cagatagage | ttcgttctga | agattctcca |
| 841 | gatattttga | catattctca | tgaaagaaaa | gttgaaaagc | ttattgttcc | gcttggtgaa |
| 901 | gaacttgcag | ccatccaaaa | gacctatatc | cagattttgg | aatcatttgc | tcgttctttg |
| 961 | attcagagga | atgttttgat | gagaagggat | atcccaaatc | taacaaaata | tcagataatt |
| 1021 | ctggcaagag | atcagtttag | gaaaaaccca | tctccgaata | ttgtgggaat | acaacaaggc |
| 1081 | ataatcgagg | gagagtttgc | tatttgtatt | agtttatatc | atggttatga | attattgcag |
| 1141 | caaatgggaa | tgagatcatt | atatttcttc | ctttgtggaa | ttatggatgg | aactaaaggg |
| 1201 | atgacacggt | caaaaaatga | acttggccga | aatgaagact | tcatgaaact | ctataatcat |
| 1261 | ctagagtgta | tgtttgcacg | tacacgtagt | acttcagcaa | atggtatttc | tgctatccaa |
| 1321 | caaggagata | aaaataaaaa | atttgtttat | agtcatccaa | agttaaagaa | attagaagaa |
| 1381 | gttgtaattg | aacacttcaa | gtcatggaat | gctgaaaaca | ctactgaaaa | gaaacgtgat |
| 1441 | gagacccgag | ttatgatctt | ctcttcattt | cgagatagtg | ttcaagaaat | tgcagaaatg |
| 1501 | ctttcacagc | atcagccaat | tattagagta | atgacttttg | tcggccatgc | ctcagggaaa |
| 1561 | agcacgaagg | gttttaccca | gaaggagcaa | ctggaggtag | tgaaacagtt | tcgtgacggt |
| 1621 | ggttacaaca | cgctggtttc | tacctgtgtg | ggtgaagaag | gtttggatat | aggagaagtt |
| 1681 | gatcttataa | tatgttttga | ttcccagaag | agcccaattc | gtcttgtaca | acgaatgggt |
| 1741 | agaactggcc | gtaaacgtca | aggcaggata | gttattatcc | tttctgaagg | acgagaggaa |
| 1801 | cgtatttata | atcagagtca | gtccaacaaa | agaagtatat | ataaagctat | ttcaagtaac |
| 1861 | aggcaggtcc | ttcattttta | ccaaagaagt | ccacgaatgg | ttcctgatgg | aatcaaccca |
| 1921 | aaattacaca | aaatgttcat | cacacatggt | gtctatgaac | cagagaagcc | ttctcggaac |
| 1981 | ttgcagcgaa | agtcatctat | cttttcctat | agggatggaa | tgaggcaaag | tagcctaaag |
| 2041 | aaagattggt | tcttatcaga | agaagaattt | aaattatgga | acagacttta | tagattaagg |
| 2101 | gacagtgatg | aaattaaaga | gataacattg | cctcaagttc | agttttcttc | tttacaaaat |
| 2161 | gaggaaaaca | aaccagctca | agaatcaacc | actggaattc | atcaactctc | tctctctgaa |
| 2221 | tggagactgt | ggcaagatca | tcctttgcct | acacatcaag | ttgatcactc | agatcgatgc |
| 2281 | cgccatttta | taggccttat | gcaaatgata | gagggaatga | gacacgaaga | gggagaatgc |
| 2341 | agctatgaat | tggaagttga | atcttattta | caaatggaag | atgttacctc | aacatttatt |
| 2401 | gctcccagga | atgaatctaa | taatcttgcc | agtgacacct | ttatcactca | caagaaatcg |
| 2461 | tcatttataa | agaacataaa | tcaaggcagt | tcatcctcag | tgatagaatc | tgatgaagaa |
| 2521 | tgtgctgaaa | ttgttaaaca | aactcatatc | aaacctacta | aaattgtttc | tttaaagaaa |
| 2581 | aaagtgtcta | aagaaataaa | aaaagatcag | cttaaaaaag | aaaataatca | cggtattata |
| 2641 | gattctgtag | ataatgacag | aaattccact | gttgaaaata | tttttcaaga | agacctacca |
| 2701 | aatgataaaa | ggacatcaga | tacagatgaa | attgctgcca | catgtactat | taatgaaaat |
| 2761 | gttattaaag | aaccgtgtgt | gttattaaca | gagtgtcagt | ttacaaataa | atccactagt |
| 2821 | tcacttgctg | gaaatgtttt | agattctggt | tataacagtt | tcaatgatga | aaaatctgtt |
| 2881 | tcatctaact | tatttcttcc | attcgaagaa | gagctttata | ttgttagaac | agatgaccaa |
| 2941 | ttttataatt | gtcactcatt | gacaaaagag | gtactagcta | atgtagagag | atttttatct |
| 3001 | tattctcctc | cgcctctcag | tggactctca | gacttggaat | atgaaattgc | taagggtact |
| 3061 | gcacttgaga | atttgctttt | cttaccctgt | gcagagcatt | tacgaagtga | taaatgcacc |
| 3121 | tgtttgctgt | cacattcagc | tgtgaattct | caacagaatt | tagaattgaa | ttcacttaaa |
| 3181 | tgtataaatt | atccatctga | aaaaagttgc | ctttatgata | tacctaatga | taatatttct |
| 3241 | gatgagccaa | gtctctgtga | ctgtgatgta | cataaacata | atcaaaatga | aaatttagta |
| 3301 | cctaacaatc | gtgttcaaat | acacagaagc | cctgcacaga | atttagttgg | agagaacaat |
| 3361 | catgatgttg | ataacagtga | cctcccagta | ttgtccactg | atcaagatga | aagtttgctg |
| 3421 | ttatttgaag | atgttaatac | agagttcgac | gatgtgagtc | tttcaccctt | gaacagtaaa |
| 3481 | agcgaatctt | tacctgtgtc | agacaaaact | gctattagtg | aaacgcctct | ggtctctcag |
| 3541 | ttcttaattt | ctgatgaact | tttgttggac | aataattctg | aactccaaga | tcaaatcacc |
| 3601 | cgtgatgcta | atagttttaa | atctcgtgat | cagagaggtg | tacaggaaga | aaaagtgaag |
| 3661 | aatcatgagg | atatttttga | ttgctctagg | gatttatttt | ctgttacctt | tgatttagga |
| 3721 | ttctgtagtc | cagattctga | tgatgaaata | ttggaacata | Catcagatag | caatagacct |
| 3781 | ctagatgatc | tatatggaag | gtatttggaa | attaaggaga | taagtgatgc | aaattatgtt |
| 3841 | tcgaatcaag | cactaatacc | aagagatcat | agtaaaaatt | ttactagtgg | aactgttatt |
| 3901 | atcccatcaa | atgaagatat | gcagaatcca | aattatgtac | atttgccact | gagtgcagca |
| 3961 | aaaaatgaag | aattgttatc | tcctggttat | tctcagtttt | ctttaccagt | gcaaaaaaaa |
| 4021 | gttatgagta | caccactctc | taaatcaaac | acattgaact | cattttctaa | gataagaaag |
| 4081 | gaaatactta | agacaccaga | ttctagtaag | gaaaaagtaa | acctacaaag | attcaaagaa |
| 4141 | gcattgaatt | caacttttga | ttattcagag | ttttctctag | aaaagtctaa | aagcagtggt |
| 4201 | ccaatgtatc | tgcataaatc | ctgtcattct | gttgaagatg | gacaattatt | aacaagtaac |
| 4261 | gaaagtgaag | atgacgagat | tttccgaaga | aaagttaaaa | gagcaaaagg | aaatgtttta |
| 4321 | aactctcctg | aggatcagaa | aaatagtgaa | gttgattctc | cacttcatgc | tgtcaaaaag |
| 4381 | cgcagatttc | ctataaacag | atcagaatta | tcatctagtg | atgagagtga | gaattttccc |
| 4441 | aaaccatgtt | cacaattaga | agacttcaag | gtttgtaacg | ggaatgccag | aagaggcatc |
| 4501 | aaagtcccaa | agagacagag | tcacttaaag | catgtagcta | ggaagttttt | agatgatgaa |
| 4561 | gcagaacttt | ctgaagaaga | tgcagaatat | gtttcatcag | atgaaaatga | tgagtcagaa |
| 4621 | aatgaacaag | attcctcatt | acttgacttt | ttaaatgatg | aaactcaact | ttcacaggct |
| 4681 | ataaatgatt | ctgaaatgag | agctatttac | atgaaatctt | tgcgtagtcc | aatgatgaac |
| 4741 | aataagtaca | aaatgattca | taagacacat | aaaaacataa | acattttctc | gcagattcct |
| 4801 | gaacaagatg | aaacctattt | agaggatagt | ttttgtgttg | atgaagagga | gtcttgcaaa |
| 4861 | ggccaatcaa | gtgaagaaga | agtttgtgtt | gattttaact | taataactga | tgattgcttt |
| 4921 | gcaaatagta | aaaagtataa | aactcgacgt | gcagtaatgc | taaaagaaat | gatggaacaa |
| 4981 | aattgtgcac | attcaaaaaa | gaaattatcc | agaattattt | taccagatga | ttcaagtgag |
| 5041 | gaggagaaca | atgtaaatga | taaaagagaa | tctaatattg | cggttaaccc | aagcactgtt |
| 5101 | aagaagaaca | aacaacagga | ccattgttta | aattcagtgc | cttctggatc | ttctgcgcag |
| 5161 | tccaaggtgc | gttctactcc | aagagttaat | ccattagcaa | agcagagcaa | acagacatcg |
| 5221 | ctgaatttaa | aggatacaat | ttccgaagtc | tcagacttca | aacctcagaa | tcataatgaa |
| 5281 | gtccagtcta | ccacaccacc | cttcactact | gttgattcac | agaaagactg | tagaaaattt |
| 5341 | ccagttccac | agaaggatgg | tagtgctttg | gaggattcta | gcacttcagg | ggcatcctgt |
| 5401 | tccaagtcaa | gaccacattt | agctgggaca | catacttctc | ttagacttcc | gcaggaagga |
| 5461 | aaaggaacct | gtattcttgt | aggtggtcat | gaaatcactt | ctggattaga | agtaatttct |
| 5521 | tccctaagag | caattcatgg | gttgcaagta | gaagtttgtc | ctcttaatgg | ctgtgattac |
| 5581 | atcgtgagta | atcgcatggt | ggtggaaagg | aggtctcaat | ctgagatgtt | aaatagtgtc |
| 5641 | aataagaaca | agttcattga | gcagatccag | cacctgcaga | gtatgtttga | aagaatatgt |
| 5701 | gtgattgtgg | aaaaggacag | agaaaaaaca | ggagacacat | caaggatgtt | taggagaaca |
| 5761 | aagagctatg | acagcctgct | gactacctta | attggcgctg | gaatccgaat | tcttttcagt |
| 5821 | tcctgccaag | aagaaaccgc | agatttgcta | aaggaactgt | ctttagtgga | acaaagaaag |
| 5881 | aatgttggta | ttcatgttcc | aacagtggtg | aatagtaata | aaagtgaggc | actccagttt |
| 5941 | tatttaagta | ttcccaatat | aagttatata | actgcattaa | atatgtgtca | ccagttttca |
| 6001 | tctgtgaaaa | ggatggctaa | cagctcactt | caagaaatct | ccatgtatgc | acaagtaact |
| 6061 | catcagaagg | ctgaggagat | ctatagatat | attcactatg | tatttgacat | acaaatgtta |
| 6121 | ccaaatgatc | ttaaccaaga | tagactgaaa | tctgatatat | aatcaagctg | ctcaagatgg |
| 6181 | ggttttcaaa | gacctctcac | aatattaaat | gcacttcaat | aatcattgct | gttttatgtt |
| 6241 | tatttgtaaa | taagagaata | ttttatttaa | atattttata | ttgtatacat | ttttatttat |
| 6301 | agattataga | aattattaaa | aaagaaaaat | ctgatgttca | gtgatcattt | tgactagatt |
| 6361 | ataaaactaa | tttttcttat | taaataaaac | aaggtttatt | aaaagtgtta | ctaaggatag |
| 6421 | tttaagaaag | taaaagctaa | gctagagata | tactttggaa | tgtttcccaa | aattaaagtt |
| 6481 | gtactgttgt | gataaatagt | aaagttgaca | tgtctatgac | tacagccaac | ttgtcgattt |
| 6541 | tccctatgtg | tagatagtat | acttttaagt | gtactgattc | taaatacatg | tacttggtaa |
| 6601 | ggtgtgggtg | atgggtgggt | tgtgagataa | atgacccagt | aactaggaaa | gtagaaaact |
| 6661 | taactgaatg | tttatctgac | caaaggtgtg | tcccagttaa | gtactgtcaa | atctattaat |
| 6721 | atgaactctg | atatggtttg | gctgtgtccc | caaccaaaat | ctcatcttga | cttgtaatct |
| 6781 | gaattataat | cccaatatat | tggggaggga | cctcctggaa | cgtgattagc | tcatgggggc |
| 6841 | ggttccccca | tgctgttcta | gtgatagttc | tcagaggatc | tgatggtttt | ataagctttt |
| 6901 | cctctgttca | ctctgcagtt | ctcttgccta | ctgccatgtg | gaaaaggaaa | cgtttgcttc |
| 6961 | ccctccacca | tgattgtaag | ttcccgaggc | ctccccagcc | atgcaggact | gtgagtcaat |
| 7021 | taaacatctt | ttccttataa | attaaaaaaa | aaaaaaaaaa | | |

SEQ ID NO: 3 (siFa)
GGATGTTTAGGAGAACAAAGAGCTA
SEQ ID NO: 4 (siFb)
CCCATCAAATGAAGATATGCAGAAT

1. A method of treatment of a cancer in an individual in need thereof comprising; reducing the expression or activity of a Fanconi anaemia, complementation group (FANC) protein in the individual, wherein the cancer is SETD2 deficient.

2. A method according to claim 1 wherein the FANC protein is a FANCM, FANCD2 or FANCI protein.

3. A method according to claim 1 or claim 2 wherein FANC expression or activity is reduced by administering a FANC antagonist to the individual.

4. A method according to claim 3 wherein the FANC antagonist is a FANCM antagonist.

5. A method according to claim 4 wherein the FANCM antagonist reduces the expression of FANCM.

6. A method according to claim 5 wherein the FANCM antagonist is a suppressor nucleic acid.

7. A method according to claim 6 wherein the suppressor nucleic acid is siRNA or shRNA.

8. A method according to claim 6 wherein the suppressor nucleic acid is an antisense oligonucleotide.

9. A method according to claim 4 wherein the FANCM antagonist is a targeted nuclease that reduces expression of FANCM.

10. A method according to claim 9 wherein the targeted nuclease is a CRISPR associated nuclease, said CRISPR associated nuclease being administered in combination with a guide RNA that recognises a target sequence within the FANCM gene.

11. A method according to claim 4 wherein the FANCM antagonist reduces the activity of FANCM.

12. A method according to claim 11, wherein the FANCM antagonist is a FANCM inhibitor.

13. A method according to claim 12, wherein the FANCM inhibitor is a small chemical molecule, optionally wherein the small chemical molecule is a non-polymeric organic compound.

14. A method according to claim 11 or 12, wherein the FANCM antagonist is an antibody, antibody fragment, antibody derivative, or non-immunoglobulin binding molecule that specifically binds to FANCM.

15. A method according to any one of claims 12 to 14, wherein the antagonist reduces ATP-dependent DNA helicase/translocase activity of FANCM, optionally wherein the antagonist binds to the DEAH helicase domain.

16. A method according to claim 3 wherein the FANC antagonist is a FANCD2 antagonist.

17. A method according to claim 16 wherein the FANCD2 antagonist reduces the expression of FANCD2.

18. A method according to claim 17 wherein the FANCD2 antagonist is a suppressor nucleic acid.

19. A method according to claim 18 wherein the suppressor nucleic acid is siRNA or shRNA.

20. A method according to claim 18 wherein the suppressor nucleic acid is an antisense oligonucleotide.

21. A method according to claim 16 wherein the FANCD2 antagonist is a targeted nuclease that reduces expression of FANCM.

22. A method according to claim 21 wherein the targeted nuclease is a CRISPR associated nuclease, said CRISPR associated nuclease being administered in combination with a guide RNA that recognises a target sequence within the FANCD2.gene.

23. A method according to claim 16 wherein the FANCD2 antagonist reduces the activity of FANCD2.

24. A method according to claim 23, wherein the FANCD2 antagonist is a FANCD2. inhibitor.

25. A method according to claim 24, wherein the FANCD2 inhibitor is a small chemical molecule, optionally wherein the small chemical molecule is a non-polymeric organic compound.

26. A method according to claim 23 or 24, wherein the FANCD2 antagonist is an antibody, antibody fragment, antibody derivative, or non-immunoglobulin binding molecule that specifically binds to FANCD2.

27. A method according to claim 3 wherein the FANC antagonist is a FANCI antagonist.

28. A method according to claim 27 wherein the FANCI antagonist reduces the expression of FANCI.

29. A method according to claim 28 wherein the FANCI antagonist is a suppressor nucleic acid.

30. A method according to claim 29 wherein the suppressor nucleic acid is siRNA or shRNA.

31. A method according to claim 29 wherein the suppressor nucleic acid is an antisense oligonucleotide.

32. A method according to claim 27 wherein the FANCI. antagonist is a targeted nuclease that reduces expression of FANCI.

33. A method according to claim 32 wherein the targeted nuclease is a CRISPR associated nuclease said CRISPR associated nuclease being administered in combination with a guide RNA that recognises a target sequence within the FANCI gene.

34. A method according to claim 33 wherein the FANCI antagonist reduces the activity of FANCI.

35. A method according to claim 34, wherein the FANCI antagonist is a FANCI inhibitor.

36. A method according to claim 35, wherein the FANCI inhibitor is a small chemical molecule, optionally wherein the small chemical molecule is a non-polymeric organic compound.

37. A method according to claim 34 or 35, wherein the FANCI antagonist is an antibody, antibody fragment, antibody derivative, or non-immunoglobulin binding molecule that specifically binds to FANCI.

38. A method according to any one of the preceding claims, wherein the SETD2 deficient cancer is selected from the group consisting of leukaemia, such as AML, CML, ALL and CLL, lymphoma, such as Hodgkin lymphoma, non-Hodgkin lymphoma and multiple myeloma, and solid cancers such as sarcomas; skin cancer; melanoma, bladder cancer; brain cancer, such as glioblastoma multiforme, such as paediatric glioblastoma multiforme; breast cancer; uterus cancer; oral cancer; ovary cancer; prostate cancer; lung cancer, such as lung adenocarcinoma, and lung squamous cell carcinoma; colorectal cancer; cervical cancer; liver cancer; head and neck cancer; oesophageal cancer; pancreas cancer; renal cancer, such as clear cell renal cancer; stomach cancer; testicular cancer; cancer of the gall bladder and biliary tracts; thyroid cancer; thymus cancer; cancer of bone; and cerebral cancer.

39. A method according to any one of the preceding claims wherein the SETD2 deficient cancer comprises one or more cancer cells with a mutation in SETD2 or loss of SETD2.

40. A method according to any one of the preceding claims, the method further comprising a step of identifying an individual as having a cancer deficient in SETD2.

41. A method according to any one of the preceding claims, the method further comprising administering a chemotherapeutic agent to the individual.

42. A FANC antagonist for use in a method of treatment of a SETD2 deficient cancer.

43. A FANC antagonist for use according to claim 42, wherein the FANC antagonist is a FANCM antagonist, FANCD2 antagonist or FANCI antagonist.

44. A FANC antagonist for use according to claim 42 or 43, wherein the method is a method of treatment according to any one of claims 1 to 41.

45. Use of a FANC antagonist in the manufacture of a medicament for treatment of a SETD2 deficient cancer.

46. Use according to claim 45 wherein the FANC antagonist is a FANCM antagonist, FANCD2 antagonist or FANCI antagonist.

47. Use according to claim 46, wherein the treatment is a method of treatment according to any one of claims 1 to 41.

48. A method of screening for a compound that induces cell death in SETD2 deficient cancer cells, comprising determining the binding of a test compound to a FANC protein, wherein binding to FANC protein is indicative that the compound induces cells death in SETD2 deficient cells.

49. A method of screening for a compound that induces cell death in SET2D deficient cancer cells comprising determining the effect of a test compound on the expression or activity of FANC protein, wherein reduction in expression or activity of FANC protein is indicative that the compound induces cell death in SETD2 deficient cancer cells.

50. A method according to claim 48 or 49 wherein the FANC protein is a FANCM protein, FANCD2 protein or FANCI protein.

51. A method according to any one of claims 48 to 50 comprising identifying the test compound as a compound which reduces the expression or activity of FANC protein.

52. A method according to claim 51, further comprising isolating or purifying the identified compound.

53. A method of determining responsiveness of a cancer in an individual to treatment with a FANC antagonist comprising determining the presence of one or more SETD2 deficient cells in a sample obtained from the individual, the presence of one or more SETD2 deficient cancer cells in the sample being indicative that the cancer is responsive to the treatment with the FANC antagonist.

54. A method of selecting an individual having a SETD2 deficient cancer for treatment with a FANC antagonist, the method comprising; identifying a cancer cell obtained from the individual as deficient in SETD2 relative to normal cells.

55. A method according to claim 53 or 54 wherein the FANC antagonist is a FANCM antagonist, FANCD2 antagonist or FANCI antagonist.

56. A method according to any one of claims 53 to 55, further comprising; providing an inhibitor of FANC suitable for administration to said individual.


SEQUENCE LISTING (4 sequences)

SEQ ID NO: 1 - protein, 2022 aa, synthetic construct
MSGRQRTLFQTWGSSISRSSGTPGCSSGTERPQSPGSSKAPLPAAAEAQLESDDDVLLVAAYEAERQLCLENGGFCTSAGALWIYPTNCPVRDYQLHISRAALFCNTLVCLPTGLGKTFIAAVVMYNFYRWFPSGKVVFMAPTKPLVTQQIEACYQVMGIPQSHMAEMTGSTQASTRKEIWCSKRVLFLTPQVMVNDLSRGACPAAEIKCLVIDEAHKALGNYAYCQAVQQVITNLLIGQIELRSEDSPDILTYSHERKVEKLIVPLGEELAAIQKTYIQILESFARSLIQRNVLMRRDIPNLTKYQIILARDQFRKNPSPNIVGIQQGIIEGEFAICISLYHGYELLQQMGMRSLYFFLCGIMDGTKGMTRSKNELGRNEDFMKLYNHLECMFARTRSTSANGISAIQQGDKNKKFVYSHPKLKKLEEVVIEHFKSWNAENTTEKKRDETRVMIFSSFRDSVQEIAEMLSQHQPIIRVMTFVGHASGKSTKGFTQKEQLEVVKQFRDGGYNTLVSTCVGEEGLDIGEVDLIICFDSQKSPIRLVQRMGRTGRKRQGRIVIILSEGREERIYNQSQSNKRSIYKAISSNRQVLHFYQRSPRMVPDGINPKLHKMFITHGVYEPEKPSRNLQRKSSIFSYRDGMRQSSLKKDWFLSEEEFKLWNRLYRLRDSDEIKEITLPQVQFSSLQNEENKPAQESTTGIHQLSLSEWRLWQDHPLPTHQVDHSDRCRHFIGLMQMIEGMRHEEGECSYELEVESYLQMEDVTSTFIAPRNESNNLASDTFITHKKSSFIKNINQGSSSSVIESDEECAEIVKQTHIKPTKIVSLKKKVSKEIKKDQLKKENNHGIIDSVDNDRNSTVENIFQEDLPNDKRTSDTDEIAATCTINENVIKEPCVLLTECQFTNKSTSSLAGNVLDSGYNSFNDEKSVSSNLFLPFEEELYIVRTDDQFYNCHSLTKEVLANVERFLSYSPPPLSGLSDLEYEIAKGTALENLLFLPCAEHLRSDKCTCLLSHSAVNSQQNLELNSLKCINYPSEKSCLYDIPNDNISDEPSLCDCDVHKHNQNENLVPNNRVQIHRSPAQNLVGENNHDVDNSDLPVLSTDQDESLLLFEDVNTEFDDVSLSPLNSKSESLPVSDKTAISETPLVSQFLISDELLLDNNSELQDQITRDANSFKSRDQRGVQEEKVKNHEDIFDCSRDLFSVTFDLGFCSPDSDDEILEHTSDSNRPLDDLYGRYLEIKEISDANYVSNQALIPRDHSKNFTSGTVIIPSNEDMQNPNYVHLPLSAAKNEELLSPGYSQFSLPVQKKVMSTPLSKSNTLNSFSKIRKEILKTPDSSKEKVNLQRFKEALNSTFDYSEFSLEKSKSSGPMYLHKSCHSVEDGQLLTSNESEDDEIFRRKVKRAKGNVLNSPEDQKNSEVDSPLHAVKKRRFPINRSELSSSDESENFPKPCSQLEDFKVCNGNARRGIKVPKRQSHLKHVARKFLDDEAELSEEDAEYVSSDENDESENEQDSSLLDFLNDETQLSQAINDSEMRAIYMKSLRSPMMNNKYKMIHKTHKNINIFSQIPEQDETYLEDSFCVDEEESCKGQSSEEEVCVDFNLITDDCFANSKKYKTRRAVMLKEMMEQNCAHSKKKLSRIILPDDSSEEENNVNDKRESNIAVNPSTVKKNKQQDHCLNSVPSGSSAQSKVRSTPRVNPLAKQSKQTSLNLKDTISEVSDFKPQNHNEVQSTTPPFTTVDSQKDCRKFPVPQKDGSALEDSSTSGASCSKSRPHLAGTHTSLRLPQEGKGTCILVGGHEITSGLEVISSLRAIHGLQVEVCPLNGCDYIVSNRMVVERRSQSEMLNSVNKNKFIEQIQHLQSMFERICVIVEKDREKTGDTSRMFRRTKSYDSLLTTLIGAGIRILFSSCQEETADLLKELSLVEQRKNVGIHVPTVVNSNKSEALQFYLSIPNISYITALNMCHQFSSVKRMANSSLQEISMYAQVTHQKAEEIYRYIHYVFDIQMLPNDLNQDRLKSDI

SEQ ID NO: 2 - RNA, 7060 nt, synthetic construct
  source 1..7060: mol_type = other RNA
tgtgcgaaggaaaccgatggggatcggaaccgtagcggttgagctgctgctgctacggatatctgacagaagccttcggtggttgtcggcctaatgagcggacggcaaagaacgctttttcagacgtggggctcaagtatctcccgatcatctgggactccgggttgcagctccggaactgagcgacctcagagccctggcagctccaaggcgcctttgccagcagcagcggaggctcagctggagtcggacgatgatgtgttgcttgtcgcggcgtacgaggctgagcggcagttgtgtctagagaatggcgggttctgcacctccgcgggcgccctgtggatttaccctaccaattgcccagtgcgggactaccagctgcacatttcccgggctgctctgttttgcaatacgctggtgtgtctgcctaccggactgggaaagacctttattgccgccgtggtcatgtacaatttctaccgctggttcccttcaggaaaggtggtcttcatggccccaacgaaacccttggtgacacagcagatcgaggcttgctaccaggtgatgggtatcccgcaatcccacatggccgaaatgacagggtctacacaagcttccaccaggaaggaaatatggtgcagtaagagagtgctttttcttacacctcaggtcatggtaaatgacctttctagaggagcttgtcccgctgctgaaataaagtgtttagttattgatgaagctcataaagctctcggaaactatgcttattgccaggctgtgcaacaagttattactaacctgctaattgggcagatagagcttcgttctgaagattctccagatattttgacatattctcatgaaagaaaagttgaaaagcttattgttccgcttggtgaagaacttgcagccatccaaaagacctatatccagattttggaatcatttgctcgttctttgattcagaggaatgttttgatgagaagggatatcccaaatctaacaaaatatcagataattctggcaagagatcagtttaggaaaaacccatctccgaatattgtgggaatacaacaaggcataatcgagggagagtttgctatttgtattagtttatatcatggttatgaattattgcagcaaatgggaatgagatcattatatttcttcctttgtggaattatggatggaactaaagggatgacacggtcaaaaaatgaacttggccgaaatgaagacttcatgaaactctataatcatctagagtgtatgtttgcacgtacacgtagtacttcagcaaatggtatttctgctatccaacaaggagataaaaataaaaaatttgtttatagtcatccaaagttaaagaaattagaagaagttgtaattgaacacttcaagtcatggaatgctgaaaacactactgaaaagaaacgtgatgagacccgagttatgatcttctcttcatttcgagatagtgttcaagaaattgcagaaatgctttcacagcatcagccaattattagagtaatgacttttgtcggccatgcctcagggaaaagcacgaagggttttacccagaaggagcaactggaggtagtgaaacagtttcgtgacggtggttacaacacgctggtttctacctgtgtgggtgaagaaggtttggatataggagaagttgatcttataatatgttttgattcccagaagagcccaattcgtcttgtacaacgaatgggtagaactggccgtaaacgtcaaggcaggatagttattatcctttctgaaggacgagaggaacgtatttataatcagagtcagtccaacaaaagaagtatatataaagctatttcaagtaacaggcaggtccttcatttttaccaaagaagtccacgaatggttcctgatggaatcaacccaaaattacacaaaatgttcatcacacatggtgtctatgaaccagagaagccttctcggaacttgcagcgaaagtcatctatcttttcctatagggatggaatgaggcaaagtagcctaaagaaagattggttcttatcagaagaagaatttaaattatggaacagactttatagattaagggacagtgatgaaattaaagagataacattgcctcaagttcagttttcttctttacaaaatgaggaaaacaaaccagctcaagaatcaaccactggaattcatcaactctctctctctgaatggagactgtggcaagatcatcctttgcctacacatcaagttgatcactcagatcgatgccgccattttataggccttatgcaaatgatagagggaatgagacacgaagagggagaatgcagctatgaattggaagttgaatcttatttacaaatggaagatgttacctcaacatttattgctcccaggaatgaatctaataatcttgccagtgacacctttatcactcacaagaaatcgtcatttataaagaacataaatcaaggcagttcatcctcagtgatagaatctgatgaagaatgtgctgaaattgttaaacaaactcatatcaaacctactaaaattgtttctttaaagaaaaaagtgtctaaagaaataaaaaaagatcagcttaaaaaagaaaataatcacggtattatagattctgtagataatgacagaaattccactgttgaaaatatttttcaagaagacctaccaaatgataaaaggacatcagatacagatgaaattgctgccacatgtactattaatgaaaatgttattaaagaaccgtgtgtgttattaacagagtgtcagtttacaaataaatccactagttcacttgctggaaatgttttagattctggttataacagtttcaatgatgaaaaatctgtttcatctaacttatttcttccattcgaagaagagctttatattgttagaacagatgaccaattttataattgtcactcattgacaaaagaggtactagctaatgtagagagatttttatcttattctcctccgcctctcagtggactctcagacttggaatatgaaattgctaagggtactgcacttgagaatttgcttttcttaccctgtgcagagcatttacgaagtgataaatgcacctgtttgctgtcacattcagctgtgaattctcaacagaatttagaattgaattcacttaaatgtataaattatccatctgaaaaaagttgcctttatgatatacctaatgataatatttctgatgagccaagtctctgtgactgtgatgtacataaacataatcaaaatgaaaatttagtacctaacaatcgtgttcaaatacacagaagccctgcacagaatttagttggagagaacaatcatgatgttgataacagtgacctcccagtattgtccactgatcaagatgaaagtttgctgttatttgaagatgttaatacagagttcgacgatgtgagtctttcacccttgaacagtaaaagcgaatctttacctgtgtcagacaaaactgctattagtgaaacgcctctggtctctcagttcttaatttctgatgaacttttgttggacaataattctgaactccaagatcaaatcacccgtgatgctaatagttttaaatctcgtgatcagagaggtgtacaggaagaaaaagtgaagaatcatgaggatatttttgattgctctagggatttattttctgttacctttgatttaggattctgtagtccagattctgatgatgaaatattggaacatacatcagatagcaatagacctctagatgatctatatggaaggtatttggaaattaaggagataagtgatgcaaattatgtttcgaatcaagcactaataccaagagatcatagtaaaaattttactagtggaactgttattatcccatcaaatgaagatatgcagaatccaaattatgtacatttgccactgagtgcagcaaaaaatgaagaattgttatctcctggttattctcagttttctttaccagtgcaaaaaaaagttatgagtacaccactctctaaatcaaacacattgaactcattttctaagataagaaaggaaatacttaagacaccagattctagtaaggaaaaagtaaacctacaaagattcaaagaagcattgaattcaacttttgattattcagaattttctctagaaaagtctaaaagcagtggtccaatgtatctgcataaatcctgtcattctgttgaagatggacaattattaacaagtaacgaaagtgaagatgacgagattttccgaagaaaagttaaaagagcaaaaggaaatgttttaaactctcctgaggatcagaaaaatagtgaagttgattctccacttcatgctgtcaaaaagcgcagatttcctataaacagatcagaattatcatctagtgatgagagtgagaattttcccaaaccatgttcacaattagaagacttcaaggtttgtaacgggaatgccagaagaggcatcaaagtcccaaagagacagagtcacttaaagcatgtagctaggaagtttttagatgatgaagcagaactttctgaagaagatgcagaatatgtttcatcagatgaaaatgatgagtcagaaaatgaacaagattcctcattacttgactttttaaatgatgaaactcaactttcacaggctataaatgattctgaaatgagagctatttacatgaaatctttgcgtagtccaatgatgaacaataagtacaaaatgattcataagacacataaaaacataaacattttctcgcagattcctgaacaagatgaaacctatttagaggatagtttttgtgttgatgaagaggagtcttgcaaaggccaatcaagtgaagaagaagtttgtgttgattttaacttaataactgatgattgctttgcaaatagtaaaaagtataaaactcgacgtgcagtaatgctaaaagaaatgatggaacaaaattgtgcacattcaaaaaagaaattatccagaattattttaccagatgattcaagtgaggaggagaacaatgtaaatgataaaagagaatctaatattgcggttaacccaagcactgttaagaagaacaaacaacaggaccattgtttaaattcagtgccttctggatcttctgcgcagtccaaggtgcgttctactccaagagttaatccattagcaaagcagagcaaacagacatcgctgaatttaaaggatacaatttccgaagtctcagacttcaaacctcagaatcataatgaagtccagtctaccacaccacccttcactactgttgattcacagaaagactgtagaaaatttccagttccacagaaggatggtagtgctttggaggattctagcacttcaggggcatcctgttccaagtcaagaccacatttagctgggacacatacttctcttagacttccgcaggaaggaaaaggaacctgtattcttgtaggtggtcatgaaatcacttctggattagaagtaatttcttccctaagagcaattcatgggttgcaagtagaagtttgtcctcttaatggctgtgattacatcgtgagtaatcgcatggtggtggaaaggaggtctcaatctgagatgttaaatagtgtcaataagaacaagttcattgagcagatccagcacctgcagagtatgtttgaaagaatatgtgtgattgtggaaaaggacagagaaaaaacaggagacacatcaaggatgtttaggagaacaaagagctatgacagcctgctgactaccttaattggcgctggaatccgaattcttttcagttcctgccaagaagaaaccgcagatttgctaaaggaactgtctttagtggaacaaagaaagaatgttggtattcatgttccaacagtggtgaatagtaataaaagtgaggcactccagttttatttaagtattcccaatataagttatataactgcattaaatatgtgtcaccagttttcatctgtgaaaaggatggctaacagctcacttcaagaaatctccatgtatgcacaagtaactcatcagaaggctgaggagatctatagatatattcactatgtatttgacatacaaatgttaccaaatgatcttaaccaagatagactgaaatctgatatataatcaagctgctcaagatggggttttcaaagacctctcacaatattaaatgcacttcaataatcattgctgttttatgtttatttgtaaataagagaatattttatttaaatattttatattgtatacatttttatttatagattatagaaattattaaaaaagaaaaatctgatgttcagtgatcattttgactagattataaaactaatttttcttattaaataaaacaaggtttattaaaagtgttactaaggatagtttaagaaagtaaaagctaagctagagatatactttggaatgtttcccaaaattaaagttgtactgttgtgataaatagtaaagttgacatgtctatgactacagccaacttgtcgattttccctatgtgtagatagtatacttttaagtgtactgattctaaatacatgtacttggtaaggtgtgggtgatgggtgggttgtgagataaatgacccagtaactaggaaagtagaaaacttaactgaatgtttatctgaccaaaggtgtgtcccagttaagtactgtcaaatctattaatatgaactctgatatggtttggctgtgtccccaaccaaaatctcatcttgacttgtaatctgaattataatcccaatatattggggagggacctcctggaacgtgattagctcatgggggcggttcccccatgctgttctagtgatagttctcagaggatctgatggttttataagcttttcctctgttcactctgcagttctcttgcctactgccatgtggaaaaggaaacgtttgcttcccctccaccatgattgtaagttcccgaggcctccccagccatgcaggactgtgagtcaattaaacatcttttccttataaattaaaaaaaaaaaaaaaaa

SEQ ID NO: 3 - RNA, 25 nt, synthetic construct
  source 1..25: mol_type = other RNA
  ncRNA 1..25: ncRNA_class = siRNA
ggatgtttaggagaacaaagagcta

SEQ ID NO: 4 - RNA, 25 nt, synthetic construct
  source 1..25: mol_type = other RNA
  ncRNA 1..25: ncRNA_class = siRNA
cccatcaaatgaagatatgcagaat